(19) United States
(12) Patent Application Publication
(10) Pub. No.: US 2023/0108487 A1
(43) Pub. Date: Apr. 6, 2023

(54) INTRAOPERATIVE LOCALISATION SYSTEMS AND METHODS

(71) Applicant: 360 Knee Systems Pty Ltd, New South Wales (AU)

(72) Inventors: Brad Miles, New South Wales (AU); Joshua Twiggs, New South Wales (AU); Willy Theodore, New South Wales (AU); Matthew Baker, New South Wales (AU)

(73) Assignee: 360 Knee Systems Pty Ltd, New South Wales (AU)

(21) Appl. No.: 17/905,682

(22) PCT Filed: Feb. 26, 2021
(86) PCT No.: PCT/AU2021/050173

§ 371 Date: Sep. 6, 2022

(30) Foreign Application Priority Data
Mar. 4, 2020 (AU) 2020900654

(51) Int. Cl. A61B 34/10; A61F 2/32; A61F 2/46; G06T 7/70; G16H 20/40; G16H 50/50

(52) U.S. Cl. CPC A61B 34/10; A61F 2/32; A61F 2/4603; G06T 7/70; G16H 20/40; G16H 50/50; A61B 2034/104; A61B 2034/105; A61B 2034/108; A61F 2002/4633; A61B 2090/364

(57) ABSTRACT

This disclosure relates to an intraoperative localisation system for total joint replacement of a joint of a patient by a surgeon, the joint being associated with a bone. The localisation system comprises: an X-ray imaging device to create a digital X-ray image of the joint and a localisation object during a total joint replacement surgery; a computer system configured to: store a surgical plan comprising a digital three-dimensional model; receive the digital X-ray image of the joint and the localisation object during the total joint replacement surgery; determine a pose of the localisation object relative to the bone or the joint, based on the digital X-ray image; assess the pose of the localisation object against the surgical plan; and provide an indication of a clinical consequence of the pose in relation to the surgical plan to the surgeon.

### CROSS-REFERENCE TO RELATED APPLICATIONS

The present application claims priority from Australian Provisional Patent Application No 2020900654 filed on 4 Mar. 2020, the contents of which are incorporated herein by reference in their entirety.

### TECHNICAL FIELD

This disclosure relates to intraoperative localisation systems and methods for assisting surgery of a joint.

### BACKGROUND

Joints between bones often deteriorate over time and need to be replaced. For example, a total hip replacement is a common surgical procedure where articulating surfaces of a hip joint affected by osteoarthritis are replaced by implant components. While a reasonable outcome can be achieved in many cases, the hip joint is complex and the total hip replacement has many parameters that can be influenced by the surgeon. For example, the surgeon can influence leg length, femoral offset, vertical and horizontal centre of rotation, acetabular inclination, acetabular anteversion, and femoral stem positioning. It is difficult for most surgeons to achieve optimal values for all these parameters.

Errors in implant component positioning, or errors in compensating for errors in implant component positioning, for example, by adjusting other cooperating implant components to compensate, can increase a number of risks associated with total hip replacements. These risks can include joint dislocation, bone fracture, change in leg length, incorrect femoral offset or loosening of the joint. Similar difficulties present themselves when replacing other joints, such as knee, shoulder and elbow joints. Therefore, systems and methods are needed that assist the surgeon to improve the quality of the joint replacement.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

This specification relates to attaching a 3D marker to an implant component or the patient and X-ray imaging the marker. The software calculates the pose of the marker from the X-ray image and updates the surgical plan accordingly.

There is described an intraoperative localisation system for total joint replacement of a joint of a patient by a surgeon, the joint being associated with a bone, the localisation system comprising:
an X-ray imaging device for application of X-ray radiation to the joint and for detecting X-ray radiation to create a digital X-ray image of the joint and a localisation object during a total joint replacement surgery;
a computer system configured to:
   store a surgical plan comprising a digital three-dimensional model;
   receive the digital X-ray image of the joint and the localisation object during the total joint replacement surgery;
   determine a pose of the localisation object relative to the bone or the joint, based on the digital X-ray image;
   assess the pose of the localisation object against the surgical plan; and
   provide an indication of a clinical consequence of the pose in relation to the surgical plan to the surgeon.

The pose may be associated with a bone pose.

The pose may be associated with implant component pose.

The system may comprise the localisation object.

The indication may comprise an updated surgical plan.

The updated surgical plan may comprise an updated digital three-dimensional model.

The computer system may be configured to determine a preoperative simulated performance metric by simulating movement of the digital three-dimensional model.

The computer system may be configured to determine an intraoperative simulated performance metric by simulating movement of the updated digital three-dimensional model.

The indication may comprise the intraoperative simulated performance metric.

The indication may comprise a comparison between the intraoperative simulated performance metric and the preoperative simulated performance metric.

The intraoperative simulated performance metric may be an indication of a risk stratification.

The risk stratification may be indicative of a risk associated with multiple predicted postoperative movements by the patient.

The risk stratification may be indicative of a risk of one or more of:
dislocation of the joint;
edge loading; and
postoperative joint pain.

Determining the pose of the localisation object may comprise detecting objects in the digital image and fitting an object model to the objects.

The localisation object may comprise an X-ray opaque two-dimensional code.

Determining the pose of the localisation object may comprise determining a pose associated with the X-ray opaque two-dimensional code.

The computer system may comprise:
at least one processor; and
at least one memory storing program code accessible by the at least one processor, and configured to cause the at least one processor to:
   store the surgical plan;
   receive the digital X-ray image of the joint and the localisation object during the total joint replacement surgery;
   determine the pose of the localisation object relative to the bone or the joint based on the digital X-ray image;
   assess the pose of the localisation object against the surgical plan; and
   providing the indication to the surgeon.

The computer system may comprise:
a first computing device comprising:
   at least one first processor; and
   at least one first memory storing program code accessible by the at least one first processor, and configured to cause the at least one first processor to:
      store the store the surgical plan;
      receive the digital X-ray image of the joint and the localisation object during the total joint replacement surgery;
      determine the pose of the localisation object relative to the bone or the joint based on the digital X-ray image; and
      assess the pose of the localisation object against the surgical plan; and
a second computing device comprising:
   at least one second processor; and
   at least one second memory storing program code accessible by the at least one second processor, and configured to cause the at least one second processor to:
      provide the indication to the surgeon.

The computing device may be configured to receive the digital X-ray image from the X-ray imaging device.

The second computing device may be configured to receive the digital X-ray image from the X-ray imaging device.

The system may comprise a display.

The indication may be provided as a visual output using the display.

Determining the pose of the localisation object may comprise identifying one or more edges of the localisation object in the digital X-ray image.

The joint may be a hip joint.

There is also described a computer-implemented method for assisting a surgeon in total joint replacement of a joint of a patient, the method comprising:
storing a surgical plan comprising a digital three-dimensional model;
receiving a digital X-ray image of the joint and a localisation object during a total joint replacement surgery;
determining a pose of the localisation object relative to a bone or the joint, based on the digital X-ray image;
assessing the pose of the localisation object against the surgical plan; and
providing an indication of a clinical consequence of the pose in relation to the surgical plan to the surgeon.

The pose may be associated with a bone pose.

The pose may be associated with an implant component pose.

The indication may comprise an updated surgical plan.

The updated surgical plan may comprise an updated digital three-dimensional model.

The method may further comprise determining a preoperative simulated performance metric by simulating movement of the digital three-dimensional model.

The method may further comprise determining an intraoperative simulated performance metric by simulating movement of the updated digital three-dimensional model.

The indication may comprise the intraoperative simulated performance metric.

The indication may comprise a comparison between the intraoperative simulated performance metric and the preoperative simulated performance metric.

The intraoperative simulated performance metric may be an indication of a risk stratification.

The risk stratification may be indicative of a risk associated with multiple predicted postoperative movements by the patient.

The risk stratification may be indicative of a risk of one or more of:
dislocation of the joint;
edge loading; and
postoperative joint pain.

Determining the pose of the localisation object may comprise detecting objects in the digital X-ray image and fitting an object model to the objects.

Determining the pose of the localisation object may comprise determining a pose associated with an X-ray opaque two-dimensional code.

The method may further comprise providing the indication as a visual output using a display.

Determining the pose of the localisation object may comprise identifying one or more edges of the localisation object in the digital X-ray image.

The joint may be a hip.

There is also described a computer-readable storage medium storing instructions that, when executed by a computing device, cause the computing device to perform the method.

### BRIEF DESCRIPTION OF DRAWINGS

Examples of the present disclosure will now be described by way of non-limiting example only, with reference to the accompanying drawings, in which:

FIG. 1 illustrates one example of an intraoperative localisation system for assisting surgery of a joint.

FIG. 2 illustrates another example of the intraoperative localisation system.

FIG. 3 illustrates a process flow diagram of a method for assisting a surgeon in total joint replacement of a joint of a patient.

FIG. 4 illustrates a postoperative joint replacement X-ray.

FIG. 5 illustrates an exemplary updated digital three-dimensional model that has been manipulated to determine a postoperative range of motion of a hip joint.

FIG. 6a illustrates a schematic line drawing 600a of a patient performing a seated flexion movement.

FIG. 6b illustrates a schematic line drawing 600b of a patient performing a standing pivot extension movement.

FIG. 7 illustrates an example indication of a intraoperative simulated performance metric.

FIG. 8 illustrates another example indication of an intraoperative simulated performance metric.

FIG. 9 illustrates another example indication of an intraoperative simulated performance metric.

FIG. 10 illustrates another example indication of an intraoperative simulated performance metric.

FIG. 11 illustrates an intraoperative X-ray during a total hip replacement surgery.

FIG. 12a illustrates a perspective view of an example digital three-dimensional model with a subsequent implant component hidden.

FIG. 12b illustrates another perspective view of the digital three-dimensional model of FIG. 12a.

FIG. 12c illustrates a third perspective view of the digital three-dimensional model of FIG. 12a.

### DESCRIPTION OF EMBODIMENTS

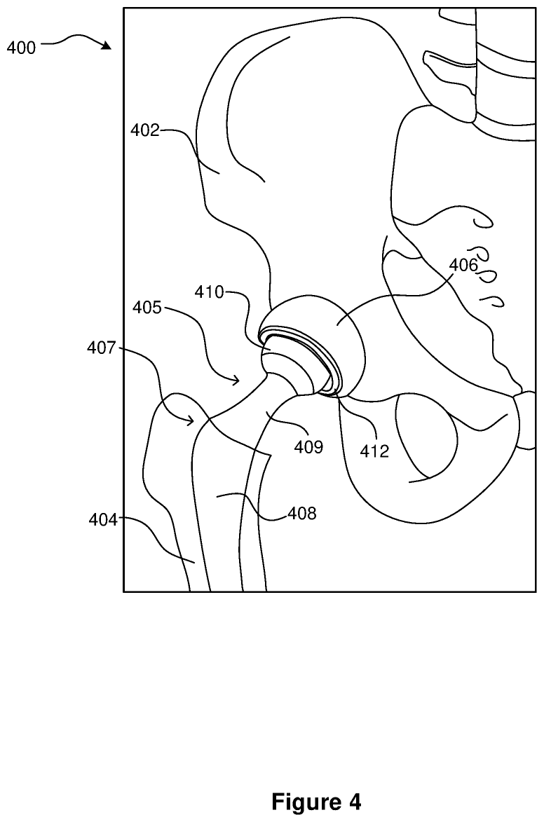

Intraoperative localisation systems and methods for assisting with surgery are described. Surgeries, such as joint replacement surgeries have many parameters that can be influenced by the surgeon. For example, FIG. 4 illustrates a postoperative joint replacement X-ray 400. An implant component assembly 405 is used in the joint replacement. The implant component assembly 405 comprises one or more implant components. In some examples, the implant component assembly 405 comprises an implant component 406. The implant component 406 can be the first implant component implanted into the patient. The implant component assembly 405 also includes a number of subsequent implant components 407. The subsequent implant components 407 are implanted after the implant component 406.

The postoperative joint replacement X-ray 400 of FIG. 4 is of a hip joint of a patient after total hip replacement. FIG. 4 shows the patient's pelvis 402, femur 404 and the implant component assembly 405. The implant component assembly 405 comprises the implant component 406 in the form of an acetabular component 406 (also “acetabular cup”). The implant component assembly 405 also comprises a plurality of subsequent implant components 407. The subsequent implant components 407 are a femoral stem 408, a neck 409, an implanted femoral head 410 and a liner 412.

During total hip replacement surgery, the surgeon removes the patient's femoral head, reams the patient's natural acetabulum with a reamer, and implants the implant component 406 (the acetabular component) in the resulting recess. The implant component 406 is a hollow hemi-spherical component. The surgeon then implants subsequent implant components 407. The liner 412 is received by the implant component 406. The liner 412 is a hollow hemi-spherical component. The liner 412 is often polymeric. The surgeon implants the femoral stem 408 in the patient's femur (such as by hammering a broach into the medullary canal), and connects the neck 409 to the femoral stem 408. The surgeon connects the implanted femoral head 410 to the neck 409. The femoral stem 408 is an elongate component. The neck 409 is an elongate component. The implanted femoral head 410 is a generally spherical component. The implant component 406 and liner 412 receive the implanted femoral head 410. The acetabular component 406, liner 412, femoral stem 408, neck 409 and implanted femoral head 410 cooperate to emulate the mechanics of a natural hip joint.

Surgeries such as total hip replacements have many parameters that the surgeon can modify. For example, in the context of the illustrated total hip replacement, the surgeon can modify leg length, horizontal centre of rotation, vertical centre of rotation, acetabular inclination, acetabular anteversion, femoral stem positioning and cement mantle thickness. In some examples, these parameters may be measured as described in Vanrusselt, Jan & Vansevenant, Milan & Vanderschueren, Geert & Vanhoenacker, Filip. (2015). “Postoperative radiograph of the hip arthroplasty: what the radiologist should know”, the contents of which is incorporated herein by reference. The disclosed intraoperative localisation systems and methods can assist with joint surgery.

System Overview

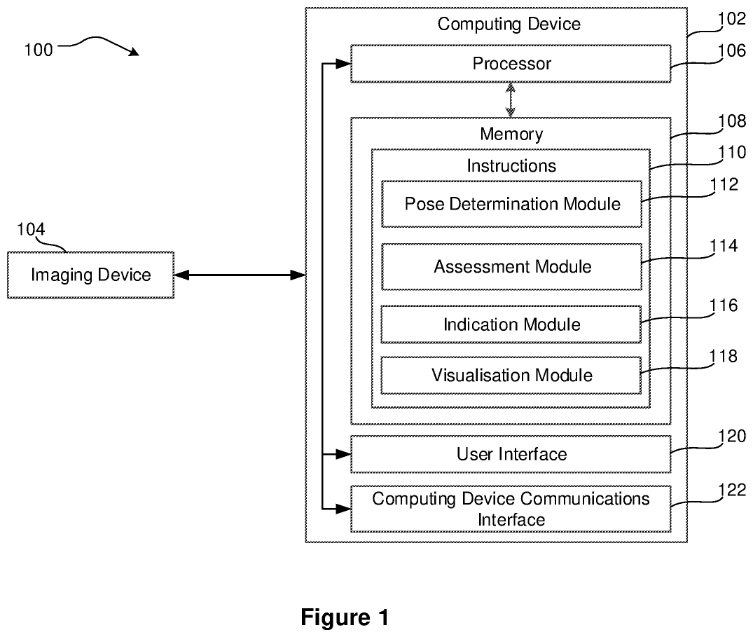

FIG. 1 illustrates an intraoperative localisation system 100 for assisting surgery of a joint. The system 100 comprises a computing device 102. The computing device 102 comprises a processor 106 and a memory 108. The system 100 also comprises an imaging device 104. The imaging device 104 is in communication with the computing device 102.

The processor 106 is configured to execute instructions 110 stored in memory 108 to cause the system 100 to function according to the described methods. The instructions 110 may be in the form of program code. The processor 106 may comprise one or more microprocessors, central processing units (CPUs), application specific instruction set processors (ASIPs), application specific integrated circuits (ASICs) or other processors capable of reading and executing instruction code.

Memory 108 may comprise one or more volatile or non-volatile memory types. For example, memory 108 may be a non-transitory compute readable medium, such as a hard drive, a solid state disk or CD-ROM. Memory 108 may comprise one or more of random access memory (RAM), read-only memory (ROM), electrically erasable programmable read-only memory (EEPROM) or flash memory. Memory 108 is configured to store program code accessible by the processor 106. The program code comprises executable program code modules. In other words, memory 108 is configured to store executable code modules configured to be executable by the processor 106. The executable code modules, when executed by the processor 106 cause the system 100 to perform the methods disclosed herein.

The computing device 102 may also comprise a user interface 120. The user interface 120 is configured to receive one or more inputs from a user. The user interface 120 is also configured to provide one or more outputs to the user. In some examples, the user can submit a request to the computing device 102 via the user interface 120, and the computing device 102 can provide an output to the user via the user interface 120. The user interface 120 may comprise one or more user interface components, such as one or more of a display device, a touch screen display, a keyboard, a mouse, a camera, a microphone, buttons, switches and lights.

The computing device 102 comprises a computing device communications interface 122. The computing device communications interface 122 is configured to facilitate communication between the computing device 102 and the imaging device 104. The computing device communications interface 122 may comprise a combination of communication interface hardware and communication interface software suitable for establishing, maintaining and facilitating communication over a relevant communication channel. In some examples, the computing device communications interface 122 is in the form of a computing device network interface.

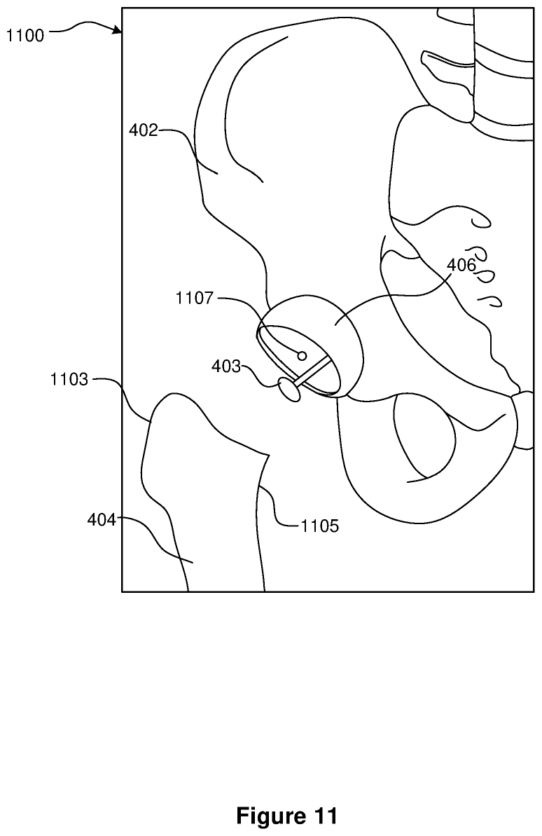

FIG. 11 illustrates an example digital X-ray image 1100. The digital X-ray image 1100 is an intraoperative X-ray image of a patient's hip. In particular, the digital X-ray image is an anterior-posterior X-ray image of the patient's hip. The digital X-ray image 1100 therefore represents an intraoperative stage of the total hip replacement surgery, with the implant component 406 (being the acetabular component) having been implanted. The digital X-ray image 1100 of FIG. 11 also illustrates the patient's pelvis 406 and the patient's femur 404. A localisation object 403 is shown. The localisation object 403 is connected to the implant component 406. The localisation object 403 extends from the implant component 406 so that it is visible in the digital X-ray image 1100.

It is to be understood that ‘image’ may refer to a two-dimensional image, such as an X-ray image stored on memory 108. In some examples, the digital X-ray image 1100 is stored in the form of a two-dimensional pixel matrix. The two-dimensional pixel matrix may comprise one intensity value for each pixel in the case of a grey scale image. Alternatively, the digital X-ray image 1100 is stored in the form of a colour model (e.g. a RGB colour model) comprising colour information of each pixel. In some examples, the colour information is carried directly by the pixel bits themselves. In some examples, the colour information is provided by a colour look-up table. The colour information may be RGB information.

However, ‘image’ may also refer to a two-dimensional projection of a three-dimensional digital model constructed from multiple two-dimensional images, such as images from a MRI or a CT scan. The surgeon can peruse this “image stack” or the two-dimensional projection on a two-dimensional screen by specifying depth values and viewing angles. Two-dimensional images and three-dimensional models may be stored on data memory 108 as multiple intensity values, such as in a two-dimensional or three-dimensional pixel matrix or as a grid model. In other examples, the two-dimensional image or the three-dimensional model is stored in a parameterised representation, such as a spline representation, and processor 106 generates a two-dimensional view on a screen (e.g. user interface 120) by interpolation based on spline parameters of the spline representation.

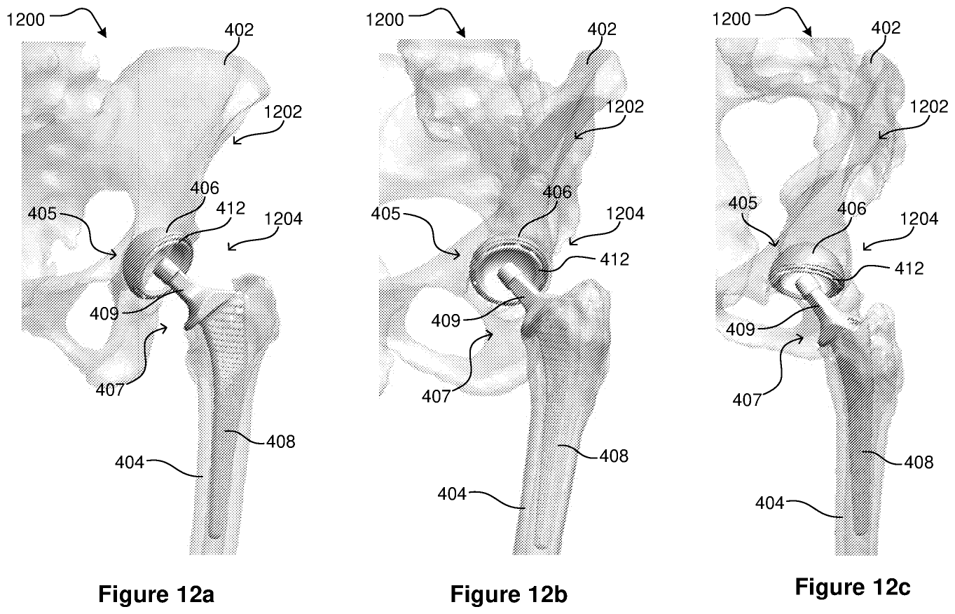

FIGS. 12a to 12c illustrate an example surgical plan. The surgical plan comprises a digital three-dimensional model 1200. In some examples the digital three-dimensional model 1200 includes details of the patient's anatomy. For example, the digital three-dimensional model 1200 can include the patient's bone and/or soft tissue structure at and around the joint that is to be replaced. The digital three-dimensional model 1200 may be a wire mesh model or finite element model. The digital three-dimensional model may represent mechanical connections for force transfer provided by the bones as well as bearing surfaces of the bones to form joints. The digital three-dimensional model 1200 can also include representation of the implant component assembly 405, including a wire mesh or finite element model of the implant component assembly 405 together with pose and 3D location and/or placement within the digital three-dimensional model 1200. As a result, the representation of the implant component assembly 405 can also represent mechanical connections for force transfer and bearing surfaces to form joints. As illustrated, a subsequent implant component 407 is hidden in FIGS. 12a to 12c. In particular, an implanted femoral head 410 is hidden. The digital three-dimensional model 1200 is described in more detail below.

As illustrated in FIG. 1, memory 108 comprises a pose determination module 112 configured to receive the digital X-ray image 1100 from the imaging device 104 and determine the pose of the localisation object relative to a bone of the joint (such as the pelvis) or the joint (such as the hip joint) based on the digital X-ray image 1100. In some examples, the digital X-ray image 1100 is a two-dimensional image. In particular, the digital X-ray image 1100 may be a fluoroscopy image or a conventional radiography image (i.e., single shot, static image). The digital X-ray image 1100 may be captured by the imaging device 104.

It is to be understood that any receiving step may be preceded by the processor 106 determining, computing and/or storing the data that is later received. For example, the processor 106 may store the data (e.g. the digital X-ray image 1100) in memory 108. The processor 106 then requests the data from memory 108, such as by providing a read signal together with a memory address. The memory 108 provides the data as a voltage signal on a physical bit line and the processor 106 receives the data. It should also be understood that any receiving step may comprise the data being received from memory 108, imaging device 104, over a network via computing device communications interface 122 and/or from another device.

Memory 108 also comprises an assessment module 114 configured to assess the pose of the implant component 406 against the surgical plan. In particular, the assessment module 114 is configured to assess the pose of the implant component 406 against the digital three-dimensional model 1200. Assessing the pose of the implant component 406 may also comprise simulating a performance metric associated with the determined placement of the implant component 406, as will be described in more detail below.

Memory 108 also comprises an indication module 116 configured to determine an indication of a clinical consequence of the current pose in relation to the surgical plan. The indication may comprise the intraoperative simulated performance metric. In particular, the indication module 116 may be configured to provide the indication of the clinical consequence, such as the intraoperative simulated performance metric as an assessment of a current placement of the implant component 406, as will be described in more detail below.

Memory 108 also comprises a visualisation module 118 configured to provide the determined indication to the surgeon. In particular, the visualisation module 118 may be configured to provide the determined indication to the surgeon by way of a visual output using the user interface 120, as will be described in more detail below.

Imaging device 104 is configured to capture the digital X-ray image 1100 of the joint and the localisation object 403 during the total joint replacement surgery. Furthermore, the imaging device 104 is configured to provide the captured digital X-ray image 1100 of the joint and the localisation object 403 to the computing device 102. In some examples, the imaging device 104 can be an X-ray imaging device (e.g. a single-shot X-ray device or a fluoroscopy device), a computed tomography (CT) imaging device, a magnetic resonance image (MRI) imaging device, a digital camera (colour or black and white) or another type of imaging device. The advantages of using an X-ray imaging device during surgery include:
Speed: Taking the images is relatively fast.
Ease of use: The device can be easily moved into place to capture the digital X-ray image 1100, e.g., on wheels, and moved out of place after capturing the digital X-ray image 1100.
Cost: X-ray imaging devices can be cheap compared to CT or MRI imaging devices.
Low radiation exposure to the patient and the surgeon.

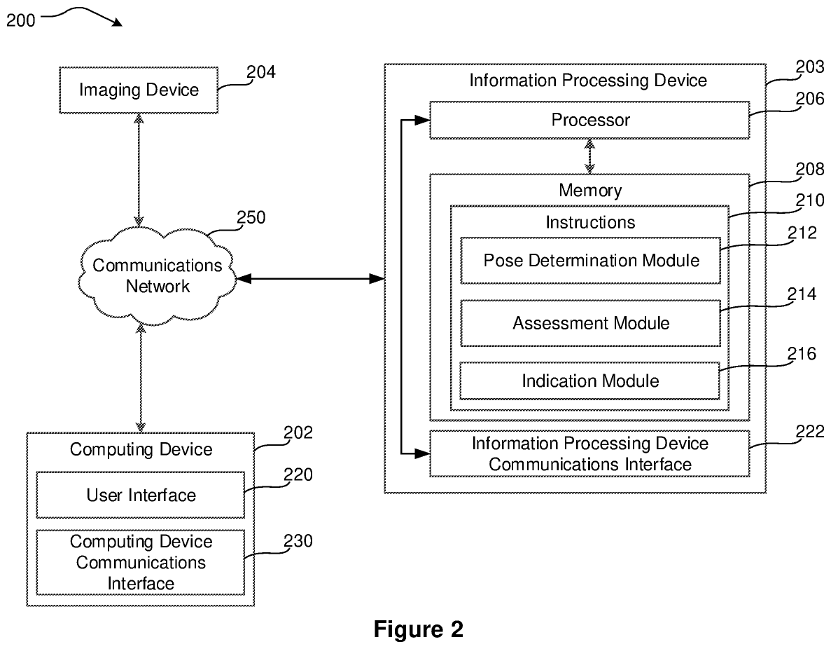

FIG. 2 illustrates another intraoperative localisation system 200 for assisting surgery of the joint. The system 200 comprises a computing device 202. The system 200 also comprises an information processing device 203. In some examples, the computing device 202 is configured to be in communication with the information processing device 203 over a communications network 250. The system 200 also comprises an imaging device 204. The imaging device 204 is configured to be in communication with the computing device 202 over the communications network 250. The imaging device 204 is configured to be in communication with the information processing device 203 over the communications network 250. Compared to FIG. 1, computing device 202 does not perform all of the data processing on the device 102 but outsources some of the processing to information processing device 203, which may be implemented as a distributed, ‘cloud’, data processing system.

The information processing device 203 comprises a processor 206. The processor 206 is configured to execute instructions 210 stored in memory 208 to cause the system 200 to perform the methods disclosed herein. The instructions 210 may be in the form of program code. The processor 206 may comprise one or more microprocessors, central processing units (CPUs), application specific instruction set processors (ASIPs), application specific integrated circuits (ASICs) or other processors capable of reading and executing instruction code. In some examples, the processor 206 may be considered a first processor.

Memory 208 may comprise one or more volatile or non-volatile memory types. For example, memory 208 may be a non-transitory compute readable medium, such as a hard drive, a solid state disk or CD-ROM. Memory 208 may comprise one or more of random access memory (RAM), read-only memory (ROM), electrically erasable programmable read-only memory (EEPROM) or flash memory. Memory 208 is configured to store program code accessible by the processor 206. The program code comprises executable program code modules. In other words, memory 208 is configured to store executable code modules configured to be executable by the processor 206. The executable code modules, when executed by the processor 206 cause the system 200 to perform the methods disclosed herein. In some examples, the memory 208 may be considered a first memory.

The information processing device 203 comprises an information processing device communications interface 222. The information processing device 203 is configured to facilitate communication between the imaging device 204 and/or the computing device 202 using the information processing device communications interface 222. The information processing device communications interface 222 may comprise a combination of communication interface hardware and communication interface software suitable for establishing, maintaining and facilitating communication over a relevant communication channel. In some examples, the information processing device communications interface 222 is in the form of an information processing device network interface. Examples of a suitable communications network 250 include a cloud server network, wired or wireless internet connection, Bluetooth® or other near field radio communication, and/or physical media such as USB. The processor 206 may receive data via the information processing device communications interface 222 and/or from memory 208.

As illustrated in FIG. 2, memory 208 comprises a pose determination module 212 configured to receive the digital X-ray image 1100 from the imaging device 204 and determine the pose of the localisation object relative to a bone of the joint (such as the pelvis) based on the digital X-ray image 1100. In some examples, the digital X-ray image 1100 is a two-dimensional image. In particular, the digital X-ray image 1100 may be a conventional radiography image or a fluoroscopy image. The digital X-ray image 1100 may be captured by the imaging device 204.

It is to be understood that any receiving step may be preceded by the processor 106 determining, computing and/or storing the data that is later received. For example, the processor 206 may store the data (e.g. the digital X-ray image 1100) in memory 208. The processor 206 then requests the data from memory 208, such as by providing a read signal together with a memory address. The memory 208 provides the data as a voltage signal on a physical bit line and the processor 206 receives the data. It should also be understood that any receiving step may comprise the data being received from memory 208, computing device 202, information processing device 203, imaging device 204, over the communications network 250 via computing device communications interface 222 and/or from another device.

Memory 208 also comprises an assessment module 214 configured to assess the pose of the implant component 406 against the surgical plan. In particular, the assessment module 114 is configured to assess the pose of the implant component against the digital three-dimensional model 1200. Assessing the pose of the implant component 406 may also comprise simulating a performance metric associated with the determined placement of the implant component 406, as will be described in more detail below.

Memory 208 also comprises an indication module 216 configured to determine an indication of a clinical consequence of the current pose in relation to the surgical plan. The indication may comprise the intraoperative simulated performance metric. In particular, the indication module 216 may be configured to provide the indication of the clinical consequence, such as the intraoperative simulated performance metric as an assessment of a current placement of the implant component 406, as will be described in more detail below.

The computing device 202 comprises the computing device communications interface 230 and the user interface 220. The computing device 202 is configured to communicate with the information processing device 203 and/or the imaging device 204 over the communications network 250 using the computing device communications interface 230. The computing device communications interface 230 may comprise a combination of communication interface hardware and communication interface software suitable for establishing, maintaining and facilitating communication over a relevant communication channel. In some examples, the computing device 202 comprises a computing device processor. The computing device processor may be considered a second processor. In some examples, the computing device 202 comprises a computing device memory. In some examples, the computing device memory may be considered a second memory. The computing device memory may store program code accessible by the computing device processor. The program code may be configured to cause the computer device processor to perform the functionality described herein.

The user interface 220 is configured to receive one or more inputs from a user. The user interface 220 is also configured to provide one or more outputs to the user. In some examples, the user can submit a request to the computing device 202 via the user interface 220, and the computing device 202 can provide an output to the user via the user interface 220. In some examples, the user interface 220 is configured to provide the indication determined by the indication module 216 by way of a visual output. The user interface 220 may comprise one or more user interface components, such as one or more of a display device, a touch screen display, a keyboard, a mouse, a camera, a microphone, buttons, switches and lights.

Imaging device 204 is configured to capture the digital X-ray image 1100 of the joint and the localisation object 403 during the total joint replacement surgery. Furthermore, the imaging device 204 is configured to provide the captured digital X-ray image 1100 of the joint and the localisation object 403 to the information processing device 203 and/or the computing device 202. In the illustrated example, the imaging device 204 is configured to transmit the digital X-ray image 1100 of the joint and the localisation object 403 to the information processing device 203 and/or the computing device 202 using the communications network 220. In some examples, the imaging device 104 can be a X-ray imaging device (e.g. a single-shot X-ray device or a fluoroscopy device), a computed tomography (CT) imaging device, a magnetic resonance image (MRI) imaging device, a digital camera (colour or black and white) or another type of imaging device as previously described.

The localisation object 403 is an object of predetermined size and shape. The localisation object 403 may have a unique two-dimensional projection (i.e. digital X-ray image) when viewed from different angles by the imaging device 104. That is, a first two-dimensional projection (first digital X-ray image) of the localisation object 403 taken by the imaging device 104 with respect to a first plane may be different to a second two-dimensional projection (second digital X-ray image) of the localisation object 403 taken by the imaging device 104 with respect to a second plane. In some examples, the first two-dimensional projection of the localisation object 403 taken with respect to the first plane is different to all other two-dimensional projections of the localisation object 403 taken with respect to all other planes that are different to the first plane. Thus, two-dimensional projections of the localisation object 403 can be used to uniquely determine the pose of the localisation object 403 with respect to the relevant viewing angle. In some examples, the localisation object 403 is configured to connect to one or more of the implant component 406, each subsequent implant component 407 and/or relevant bones, such as the pelvis 402 and/or the femur 404.

### Exemplary Embodiment

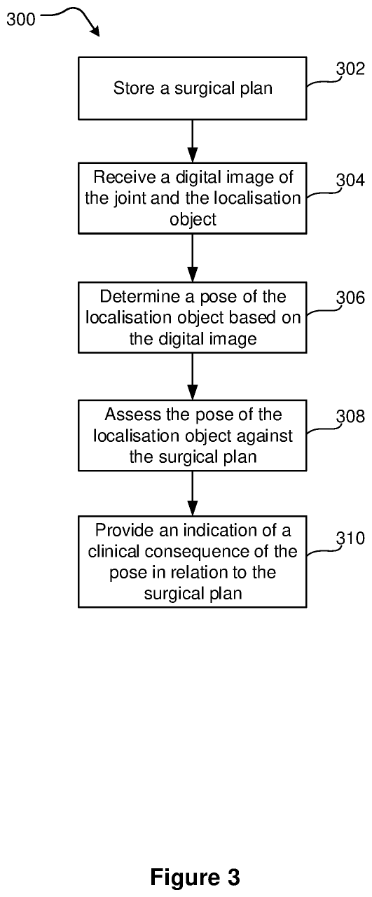

FIG. 3 illustrates a process flow diagram of a computer-implemented method 300 for assisting surgery of a joint, according to some examples. In some examples, the method 300 is performed by the intraoperative localisation system 100, as will be described in more detail below. In some examples, the method 300 is performed by the intraoperative localisation system 200, as will be described in more detail below.

FIG. 3 is to be understood as a blueprint for a software program and may be implemented step-by-step, such that each step in FIG. 3 may, for example, be represented by a function in a programming language, such as C++ or Java. The resulting source code is then compiled and stored as computer executable instructions 110, 210 on memory 108 in the case of system 100, and on memory 208 in the case of system 200.

Prior to commencing total joint replacement surgery, it can be beneficial to determine the surgical plan. In some examples, the digital three-dimensional model 1200 can represent the surgical plan. A surgeon can adjust implant component sizing and pose relative to the patient's anatomy in the digital three-dimensional model 1200, and use the model as a baseline to monitor intraoperative surgical progress.

Method 300 Performed by System 100

In some examples, the computing device 102 generates the surgical plan. That is, the computing device 102 generates the digital three-dimensional model 1200. In some examples, another computing device generates the surgical plan. That is, another computing device generates the digital three-dimensional model 1200. The digital three-dimensional model 1200 is a digital model. The digital three-dimensional model 1200 may be a hip, knee, shoulder, elbow or another joint. The digital three-dimensional model 1200 comprises an anatomical three-dimensional model 1202. The anatomical three-dimensional model 1202 is a three-dimensional model of the patient's anatomy. In particular, the anatomical three-dimensional model 1202 is a three-dimensional model of the joint to be replaced in the joint replacement surgery. In some examples, the anatomical three-dimensional model 1202 is a three-dimensional model of the patient's pre-operative anatomy. The anatomical three-dimensional model 1202 may be modified to represent the patient's anatomy after the surgery (their postoperative anatomy). For example, in cases where the patient's bone is to be cut during the surgery, the cut(s) can be included in the representation of the bone in the anatomical three-dimensional model 1202. In some examples, the anatomical three-dimensional model 1202 includes both a pre-operative anatomical three-dimensional model and postoperative anatomical three-dimensional model. In said examples, the user of the system 100 may be able to toggle between the pre-operative anatomical three-dimensional model and postoperative anatomical three-dimensional model.

Computing device 102 (or a different computing device) generates the digital three-dimensional model 1200 using information provided by a preoperative imaging device. The preoperative imaging device can be a CT imaging device or an MRI imaging device, for example.

In some examples, the preoperative imaging device is configured to provide the information to the processor 106. The processor 106 processes the information provided by the preoperative imaging device to generate the anatomical three-dimensional model 1202. The anatomical three-dimensional model 1202 is then stored in memory 108.

In some examples, a model generating computing device (not shown) processes the information provided by the preoperative imaging device to generate the anatomical three-dimensional model 1202. In said examples, the anatomical three-dimensional model 1202 is provided to the computing device 102. The anatomical three-dimensional model 1202 is then stored in memory 108.

In some examples, the digital three-dimensional model 1200 also comprises an implant component assembly three-dimensional model 1204. The implant component assembly three-dimensional model 1204 is a digital model. The implant component assembly three-dimensional model 1204 is a three-dimensional representation of the implant component assembly 405. For example, the implant component assembly three-dimensional model 1204 can comprise three-dimensional models of the implant component 406, and the one or more subsequent implant components 407. The implant component 406 can be in the form of the acetabular component 406 as previously described. The subsequent implant components 407 can be in the form of the femoral stem 408, neck 409, implanted femoral head 410 and liner 412 as previously described.

In some examples, the digital three-dimensional model 1200 represents the intended joint configuration after the surgery. That is, the implant component assembly three-dimensional model 1204 is positioned with respect to the anatomical three-dimensional model 1202 such that the digital three-dimensional model 1200 represents the intended joint configuration after the surgery. In that respect, the digital three-dimensional model 1200 can be considered a surgical plan.

The digital three-dimensional model 1200 can be transformed, such as rotated, translated and/or scaled, to correspond with the actual sizing of the patient's anatomy and the implant component assembly 405. That is, a measurement between a first point and a second point of the anatomical three-dimensional model 1202 and/or the implant component assembly three-dimensional model 1204 can be the same as a measurement between a corresponding first point and a corresponding second point of the patient's anatomy and/or the implant component assembly 405.

The implant component assembly 405, and therefore, each implant component 406 and/or each subsequent implant component 407 can be provided in a plurality of sizes. For example, each of the acetabular component 406, liner 412, femoral stem 408, neck 409 and/or femoral head 410 used in the total hip replacement illustrated in FIG. 4 can be provided in a plurality of sizes. The size of each implant component 406 and/or each subsequent implant component 407 can be determined in the digital three-dimensional model 1200.

In some examples of the digital three-dimensional model 1200, the pose of each implant component 406 and/or each subsequent implant component 407 and/or the implant component assembly 405 is determined manually. That is, a user of the system 100 can observe the patient's anatomy and/or the anatomical three-dimensional model 1202, and select a pose for each implant component 406 and/or each subsequent implant component 407 in the digital three-dimensional model 1200.

In some examples, the computing device 102 automatically determines the size of each implant component 406, each subsequent implant component 407 and/or the implant component assembly 406 of the digital three-dimensional model 1200. The determined size of each implant component 406 and/or each subsequent implant component 407 may be optimized based on anatomical geometry of the patient.

In some examples, the computing device 102 automatically determines the pose of each implant component 406 and/or each subsequent implant component 407. The determined pose of each implant component 406 and/or each subsequent implant component 407 may be optimized based on anatomical geometry of the patient.

When in the context of the total hip replacement, the digital three-dimensional model 1200 can include the patient's pelvis 406 and femur 404. The implant components used in the total hip replacement, as illustrated in FIG. 4, comprise the acetabular component 406, the liner 412, the femoral stem 408, neck 409 and the implant femoral head 410. Thus, the implant component assembly three-dimensional model 1204 for the total hip replacement can include three-dimensional representations of the acetabular component 406, liner 412, the femoral stem 408, neck 409 and/or the implant femoral head 410 to be used in the surgery.

In some examples, the computing device 102 processes the digital three-dimensional model 1200. In some examples, the model generating computing device, or another computing device processes the digital three-dimensional model 1200 and transmits the processed digital three-dimensional model 1200 to the computing device 102.

Processing the digital three-dimensional model 1200 may comprise determining one or more digital three-dimensional model parameters. The digital three-dimensional model parameters may comprise locations of one or more three-dimensional model landmarks. The three-dimensional model landmarks may be, in the case of a total hip replacement, the patient's greater trochanter 1103, lesser trochanter 1107, femoral stem alignment, femoral shaft alignment and/or the centre of rotation of the implanted femoral head 1107. The three-dimensional model landmarks may comprise a number of pelvic landmarks, for example, the anterior superior iliac spine, anterior inferior iliac spine, pubic symphysis, obturator foramen, acetabular floor, sacrum, coccyx and/or greater sciatic notch. The three-dimensional model landmarks may comprise a number of femoral landmarks, for example, the piriformis fossa and/or intertrochanteric ridge. Each three-dimensional model landmark may have an associated landmark location. Each landmark location may be a Cartesian coordinate in the reference frame of the digital three-dimensional model 1200. One or more of the three-dimensional model parameters may be associated with the implant component 406. In some examples, one or more of the three-dimensional model parameters may be indicative of a placement, pose, size and/or shape of the implant component 406.

The one or more three-dimensional model parameters may comprise one or more three-dimensional model measurements. The three-dimensional model measurements are indicative of a distance between two or more three-dimensional model landmarks. The three-dimensional model measurements may be, for example, leg length, acetabular inclination, acetabular anteversion and/or cement mantle thickness, femoral offset, anterior offset, stem varus/valgus angle, and/or the distance between one or more of the landmarks previously described.

Although the digital three-dimensional model is described as being processed after generation, in some examples, each of the anatomical three-dimensional model 1202 and/or the implant component assembly three-dimensional model 1204 are processed before being used to generate the digital three-dimensional model 1200. Thus, the digital three-dimensional model parameters may comprise anatomical three-dimensional model parameters. The anatomical three-dimensional model parameters may be determined from the anatomical three-dimensional model 1202. Furthermore, the digital three-dimensional model parameters may comprise implant component assembly three-dimensional model parameters. The implant component assembly three-dimensional model parameters may be determined from the implant component assembly three-dimensional model 1204.

At 302, the computing device 102 stores the surgical plan in memory 108. That is, the computing device 102 stores the processed digital three-dimensional model 1200 in memory 108. The computing device 102 stores the digital three-dimensional model 1200, and the associated digital three-dimensional model parameters.

The imaging device 104 captures the digital X-ray image 1100 of the joint and the localisation object 403. In particular, the imaging device 104 captures the digital X-ray image 1100 of the joint and the localisation object 403 during the total joint replacement surgery. The digital X-ray image 1100 may also comprise the implant component 406 and/or at least one subsequent implant component 407.

At 304, the computing device 102 receives the digital X-ray image 1100 of the joint and the localisation object 403. The processor 106 stores the digital X-ray image 1100 of the joint and the localisation object 403 in memory 108.

The computing device 102 processes the digital X-ray image 1100. Processing the digital X-ray image 1100 may comprise determining one or more digital image parameters. The one or more digital image parameters may comprise locations of one or more digital image landmarks. The digital image landmarks may be, in the case of a total hip replacement, the patient's greater trochanter 1103, lesser trochanter 1105, femoral stem alignment, femoral shaft alignment and/or the centre of rotation of the implanted femoral head 1107. The digital image landmarks may comprise a number of pelvic landmarks, for example, the anterior superior iliac spine, anterior inferior iliac spine, pubic symphysis, obturator foramen, acetabular floor, sacrum, coccyx and/or greater sciatic notch. The digital image landmarks may comprise a number of femoral landmarks, for example, the piriformis fossa and/or intertrochanteric ridge. Each digital image landmark may have a determined digital image landmark location. The digital image landmark location may be a Cartesian coordinate in the reference frame of the digital X-ray image 1100.

The digital image parameters may comprise one or more digital image measurements. The digital image measurements are indicative of a distance between two or more digital image landmarks. The digital image measurements may be, for example, leg length, acetabular inclination, acetabular anteversion and/or cement mantle thickness, femoral offset, anterior offset, stem varus/valgus angle, and/or the distance between one or more of the landmarks previously described.

One or more of the digital image parameters may correspond with one or more of the digital three-dimensional model parameters. Therefore, one or more of the digital image landmarks may correspond with a respective three-dimensional model landmark. Furthermore, one or more of the digital image measurements may correspond with a respective three-dimensional model measurement.

In some examples, processing the digital X-ray image 1100 may comprise scaling the digital X-ray image 1100. The digital X-ray image 1100 may be scaled using the localisation object 403. The size of the localisation object 406 is known. Thus, the digital X-ray image 1100 can be scaled to correspond with the digital three-dimensional model 1200 using a measured localisation object 403 dimension. The measured localisation object 403 dimension can, for example, be a radius of the localisation object 403 or a thickness of the localisation object 403. In some examples, the reference object may be separate from the implant component assembly 405. That is, the reference object may be unrelated to the implant component assembly 405 and/or the localisation object 403.

In some examples, the digital X-ray image 1100 may be scaled based on a comparison between one or more of the digital image parameters and one or more of the three-dimensional model parameters. In said examples, the digital X-ray image 1100 is scaled such that the relevant digital image parameter corresponds with the respective three-dimensional model parameters. Alternatively, the magnification can be calculated based on the distance between the observed object (e.g. the joint) and the imaging device 104. For example, where the distance between the joint and an X-ray origin of the imaging device 104 (i.e. the location of the imaging device 104 from which X-rays are emitted) is known, and where the distance between the joint and an X-ray detector of the imaging device 104 (i.e. the location of the imaging device that detects the X-rays) is known, the magnification of the digital X-ray image 1100 can be determined.

In some examples, processing the digital X-ray image 1100 comprises detecting one or more edges in the digital X-ray image 1100. For example, the computing device 102 detects the edges of the localisation object 403. The computing device 102 may detect the edges using a suitable edge detection method, such as using a Sobel operator.

In some examples, processing the digital X-ray image 1100 comprises detecting one or more objects in the digital X-ray image 1100. For example, an anatomical features, e.g. a bone, may be detected in the digital X-ray image 1100. Furthermore, one or more of the implant component 406 and/or the subsequent implant components 407 may be detected in the digital X-ray image 1100. In particular, the implant component 406 may be detected in the digital X-ray image 1100. Alternatively, the localisation object 403 can be detected in the digital X-ray image 1100.

In some examples, the computing device 102 detects the objects (e.g. the localisation object 403) in the digital X-ray image 1100. The computing device 102 may use the detected edges to detect the objects. Alternatively, the computing device 102 may use other features of the digital X-ray image 1100 to detect the objects. The computing device 102 may detect the objects using a suitable object detection method. For example the computing device 102 may use a machine learning method to detect the objects. In some examples, the computing device 102 detects features using the Viola-Jones object detection framework based on Haar features, a scale-invariant feature transform or a histogram of oriented gradients, and uses a classification technique such as a support vector machine to classify the objects.

In some examples, the computing device 102 is also configured to determine the pose of the objects in the digital X-ray image 1100. For example, after detecting the implant component 406, the computing device 102 is configured to determine the pose of the implant component 406. The pose of the implant component 406 may comprise an indication of the location and orientation of the implant component 406.

In some examples, the computing device 102 may be configured to use the detected edges, objects and/or poses of said objects to determine the one or more digital image parameters.

In some examples, the computing device 102 compares one or more of the digital image parameters to one or more parameter thresholds. The parameter thresholds can be indicative of the desired surgical parameters, or acceptable surgical parameters. For example, in the case of the total hip replacement, a parameter threshold can be an implant component inclination angle threshold of 40°. That is, the desired inclination angle of the implant component is 40°. The inclination angle of the implanted implant component can be determined from the digital X-ray image 1100 as previously described, and this can be compared to the implant component inclination angle threshold. In some examples, implant component inclination angle threshold is a range, for example, between 30° and 50°. The surgeon may specify the parameter thresholds, which may be selected to maximise the postoperative performance of the joint. Alternatively, the computing device 102 can automatically determine the parameter thresholds. If the implant component 406 is determined to deviate from its corresponding parameter thresholds, it can be classified as high risk.

In some examples, the parameter thresholds are equal to the desired surgical parameters. In other examples, the parameter thresholds are threshold ranges centred upon, or including the desired surgical parameter.

At 306, the computing device 102 determines the pose of the localisation object 403. The computing device 102 determines the pose of the localisation object 403 relative to the bone or the joint based on the digital X-ray image 1100. In particular, the pose determination module 112 determines the pose of the localisation object 403. The pose of the localisation object 403 is determined relative to the bone or the joint based on the digital X-ray image 1100.

The computing device 102 may have stored an object model of the localisation object 403. That is, memory 108 may comprise the stored object model. The computing device 102 attempts to match the object model against objects identified in the digital X-ray image 1100. The computing device 102 may, for example, determine one or more two-dimensional object projections of a three-dimensional object model, and compare the two-dimensional object projections to the objects detected in the digital image. The computing device 102 fits a localisation object model to the localisation object 403. The computing device 102 then determines the position and pose of the localisation object 403.

The imaging device 104 may be fixed in relation to the operating table, with a known viewing angle and distance. Thus, the computing device 102 can calculate the position and pose of the localisation object 403 in relation to this global reference frame. Processor 106 may implement feature detection using Haar-like features is disclosed in Viola and Jones, “Rapid object detection using a boosted cascade of simple features”, Computer cool Vision and Pattern Recognition, 2001, which is incorporated herein by reference.

In another example, the computing device 102 is configured to detect one or more bones in the digital X-ray image 1100. For example, the computing device 102 is configured to detect the femur 404 and/or the pelvis 402. The computing device 102 may detect the one or more bones using a bone model. The computing device 102 may attempt to match the bone model against objects identified in the digital X-ray image 1100 to detect the one or more bones as described with reference to the object model.

In another example, the computing device 102 is configured to detect the implant component 406 and/or one or more of the subsequent implant components 407. The computing device may detect the implant component 406 and/or one or more of the subsequent implant components 407 using an implant component object model. The computing device 102 may attempt to match the implant component object model against objects identified in the digital X-ray image 1100 to detect the implant component 406 and/or one or more of the subsequent implant components 407 as described with reference to the object model.

Once the bone, implant component 406 or subsequent implant component 407 is identified in the digital X-ray image 1100, the computing device 102 can determine the relative position and pose of the bone, implant component 406 or subsequent implant component 407 in relation to the localisation object 403. The relative position may be expressed as three offset angles and three translation values. Furthermore, as the location and pose of the localisation object 403 is known in the global reference frame, the location and pose of each of the implant component 406, subsequent implant component 407 and/or bone can be determined.

In some examples, the localisation object 403 is directly connected to the implant component 406. The localisation object 403 can be connected to the implant component 406 at a fixed position. The connection can be a rigid connection. This is the case illustrated in FIG. 4. The processor 106 has available a fixed spatial relationship between the localisation object 403 and the implant component 406, such as three offset angles and three offset coordinates. Thus, by determining the position and pose of the localisation object 403, the computing device 102 can determine the corresponding position and pose of the implant component 406. The pose of the localisation object 403 can therefore be considered to be associated with an implant component pose. This can advantageously be done without the computing device 102 having to detect the implant component 406 in the digital X-ray image 1100, reducing computational requirements of the computing device 102.

In some examples, the localisation object 403 is directly connected to the subsequent implant component 407. The localisation object 403 can be connected to the subsequent implant component 407 at a fixed position. The connection can be a rigid connection. The processor 106 has available a fixed spatial relationship between the localisation object 403 and the subsequent implant component 406, such as three offset angles and three offset coordinates. Thus, by determining the position and pose of the localisation object 403, the computing device 102 can determine the corresponding position and pose of the subsequent implant component 407. The pose of the localisation object 403 can therefore be considered to be associated with a subsequent implant component pose. This can advantageously be done without the computing device 102 having to detect the subsequent implant component 407 in the digital X-ray image 1100, reducing computational requirements of the computing device 102.

In some examples, the localisation object 403 is directly connected to the relevant bone. For example, the localisation object 403 may be directly connected to the femur 404. Alternatively, the localisation object 403 may be directly connected to the pelvis 402. The localisation object 403 can be connected to the relevant bone at a fixed position. The connection can be a rigid connection. The processor 106 has available a fixed spatial relationship between the localisation object 403 and the bone, such as three offset angles and three offset coordinates. Thus, by determining the position and pose of the localisation object 403, the computing device 102 can determine the corresponding position and pose of the bone (e.g. the femur 404 or the pelvis 402). The pose of the localisation object 403 can therefore be considered to be associated with a bone pose. This can advantageously be done without the computing device 102 having to detect the bone in the digital X-ray image 1100, reducing computational requirements of the computing device 102.

In some examples, the implant component 406 may comprise the localisation object 403. Alternatively, the implant component 460 may be the localisation object 403. For example, the implant component 406 may comprise an X-ray opaque two-dimensional code on a surface of the implant component 406. The X-ray opaque two-dimensional code may be a two-dimensional binary pattern with opaque and transmissive areas. The two-dimensional pattern enables determination of 6D information (three positions and three angles). The code may be a WhyCon, ARTags, April Tag, WhyCode. The code may be an Aruco code and processor 106 may execute an Aruco library available at https://docs.opencv.org/trunk/d9/d6a/group_aruco.html. This enables processor 106 to identify the location and pose of the localisation object 403 without object detection, which may make the process more robust. There may be multiple codes affixed to localisation object 403 to further improve the pose estimation. Again, the pose and position of localisation object 403 may be in relation to the bone, or in relation to the joint. In another example, a further marker, such as a further Aruco code is attached to the bone at a predefined landmark to support the detection of the bone in the image data. For example, the further Aruco code may be attached to the pelvis 402 and/or femur 404.

At 308, the computing device 102 assesses the pose of the localisation object 403 against the surgical plan. In some examples, the computing device 102 may assess the pose of the implant component 406 against the surgical plan. As the pose of the implant component 406 was determined using the determined pose of the localisation object 403, this can be considered an assessment of the pose of the localisation object 403 against the surgical plan. In some examples, the computing device 102 may assess the pose of the subsequent implant component 407 against the surgical plan. As the pose of the subsequent implant component 407 was determined using the determined pose of the localisation object 403, this can be considered an assessment of the pose of the localisation object 403 against the surgical plan. In some examples, the computing device 102 may assess the pose of the bone (e.g. the pelvis 404 or femur 402) against the surgical plan. As the pose of the bone was determined using the determined pose of the localisation object 403, this can be considered an assessment of the pose of the localisation object 403 against the surgical plan. Although described below with reference to the implant component 406, it will be appreciated that the description can also be applied to the subsequent implant component 407 and/or the bone.

In some examples, the computing device 102 determines one or more differences between the pose of the implant component 406 as represented in the digital X-ray image 1100 (and determined using the localisation object 403), and the pose of the implant component 406 of the digital three-dimensional model 1200.

The computing device 102 may determine an updated digital three-dimensional model. The computing device 102 updates the pose of the implant component 406 in the digital three-dimensional model 1200 to reflect the actual implanted pose of the implant component 406 determined from the digital X-ray image 1100 using the localisation object 403. Thus, the digital three-dimensional model 1200 is intraoperatively updated to reflect the state of the surgery at the time the digital X-ray image 1100 was captured. Updating the pose of the implant component 406 may comprise, for example, translating and/or rotating the implant component 406 of the digital three-dimensional model 1200. The computing device 102 updates the digital three-dimensional model 1200 based on the determined placement of the implant component 406 in the digital X-ray image 1100 in relation to the digital three-dimensional model 1200, thereby determining the updated digital three-dimensional model. A placement may be considered to comprise a position (e.g. a coordinate in a reference frame) and a pose (e.g. an indication of an angle within the coordinate frame).

The computing device 102 determines an intraoperative simulated performance metric by simulating movement of the digital three-dimensional model based on the placement of the implant component 406 (determined using the localisation object 403) in the digital X-ray image 1100. In particular, the performance metric simulation module 114 determines the intraoperative simulated performance metric by simulating movement of the updated digital three-dimensional model.

The computing device 102 determines the intraoperative simulated performance metric by performing a kinematic analysis on the updated digital three-dimensional model. The kinematic analysis can comprise moving the relevant portions of the updated digital three-dimensional model to determine a postoperative range of motion of the joint. This movement is performed by the computing device 102 and comprises moving elements of the digital three-dimensional model 1200, such as moving bones against each other. This movement may be defined by the shape and location of bearing surfaces of joints represented by the updated digital three-dimensional model.

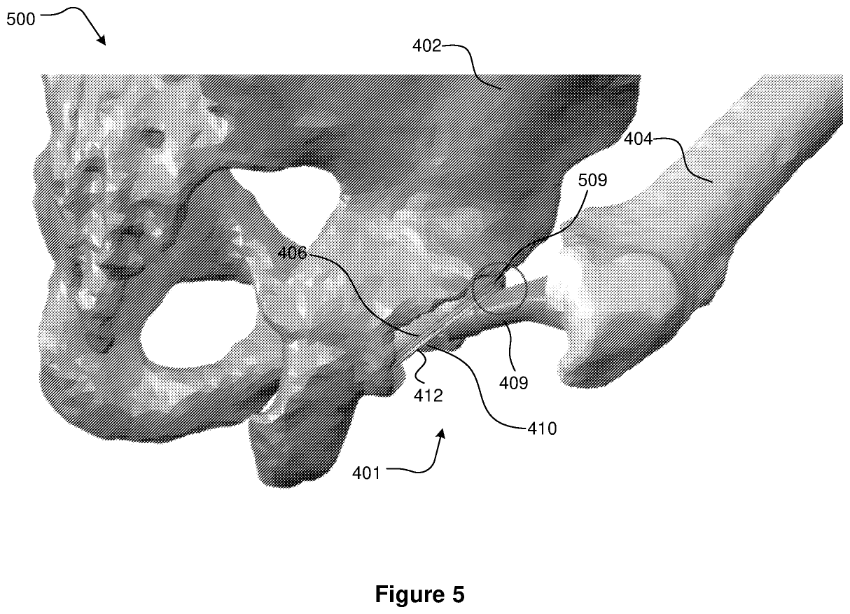

FIG. 5 illustrates an example updated digital three-dimensional model 500 that has been manipulated to determine a postoperative range of motion of a hip joint 501. In particular, updated three-dimensional model 500 of FIG. 5 has been manipulated to simulate seated flexion of the hip joint 501. Included in the updated three-dimensional model 500 is the pelvis 402, femur 404, femoral stem 408, neck 409, implant femoral head 410, liner 412 and acetabular component 406. In the example illustrated in FIG. 5, each surface of the updated digital three-dimensional model 500 is considered solid, and thus a surface of one component contacting another is indicative of a maximum range of motion in that direction. Such an impingement 509 is illustrated in FIG. 5.

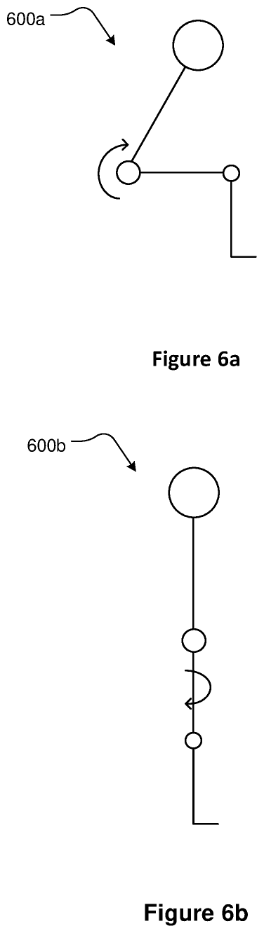

The kinematic analysis may comprise a number of postoperative joint movements. Each postoperative joint movement can simulate a typical movement of the patient after the surgery. For example, in the case of the total hip replacement, the kinematic analysis may comprise the seated flexion movement as shown in FIG. 5. FIG. 6a illustrates a schematic line drawing 600a of a patient performing a seated flexion movement. This movement occurs when the patient rotates their upper body (and thus their pelvis) forward with respect to their femoral head when in a sitting position. This movement also occurs when the patient is in the sitting position and brings their knee upwards towards their torso.

Also in the case of the total hip replacement, the kinematic analysis can comprise a standing pivot extension movement. FIG. 6b illustrates a schematic line drawing 600b of a patient performing a standing pivot extension movement. This movement occurs when the patient is standing and rotates their leg outwards about its longitudinal axis.

The kinematic analysis is associated with at least one kinematic analysis target parameter. Each kinematic analysis target parameter can be indicative of a desired or target performance of the joint. For example, the kinematic analysis target parameter can be an angle representing a target rotation desired of the joint before an impingement occurs. The computing device 102 is configured to provide a risk stratification based on a comparison between the kinematic performance of the updated digital three-dimensional model and the at least one kinematic analysis target parameter.

In some examples, a flexion target parameter can be associated with the seated flexion movement of the kinematic analysis. The flexion target parameter is indicative of a maximum flexion angle achievable by the updated digital three-dimensional model. Furthermore, an extension rotation target parameter can be associated with the standing pivot extension of the kinematic analysis. The extension rotation target is indicative of a maximum rotation angle that the femur can be rotated about the relevant leg's longitudinal axis achievable by the updated digital three-dimensional model.

The computing device 102 may also compare a current (i.e. intraoperative) implant component pose with a number of alternative poses (e.g. of the acetabular component) by determining an alternative simulated performance metric associated with an alternative implant component pose. In other words, the computing device 102 may compare an intraoperative implant component pose with the number of alternative poses. The computing device 102 can adjust the pose of the implant component 406 in the updated digital three-dimensional model, and re-run the kinematic analysis. The computing device 102 is configured to provide an alternative risk stratification based on a comparison between the kinematic performance of the updated digital three-dimensional model with the alternative implant component pose and the at least one kinematic analysis target parameter. For example, the computing device 102 can change the acetabular inclination angle of the implant component 406, and re-run the kinematic analysis. In some examples, this can be used to assist the surgeon in determining whether or not the implant component 406 that has been implanted should be removed, and/or re-implanted in a different position.

In some examples, the computing device 102 also determines the alternative simulated performance metric associated with an alternative subsequent implant component 407′. As previously described, the updated digital three-dimensional model includes one or more subsequent implant components 407 that are to be implanted after the implant component 406. The positioning of the implant component 406 that has been implanted may however mean the originally planned subsequent implant components 407 are unsuitable. Thus, the computing device 102 determines the alternative simulated performance metric associated with the alternative subsequent implant component 407′. The alternative simulated performance metric can be compared to the intraoperative simulated performance metric to assess surgical options. In some examples, this can be used to assist the surgeon in intraoperatively determining appropriate sizing for the subsequent implant components 407.

The computing device 102 determines the alternative subsequent implant component 407′. The computing device 102 can substitute the alternative subsequent implant component 407′ for the subsequent implant component 407 in the updated digital three-dimensional model, and re-run the kinematic analysis. The computing device 102 is configured to provide an alternative risk stratification based on a comparison between the kinematic performance of the updated digital three-dimensional model with the subsequent implant component 407 and the alternative subsequent implant component 407′ using the kinematic analysis target parameter.

In some examples, the computing device 102 determines a preoperative simulated performance metric. The computing device 102 determines the preoperative simulated performance metric by simulating movement of the digital three-dimensional model 1200 according to a surgical plan. In some examples, the surgical plan is the digital three-dimensional model 1200. In some examples, the surgical plan comprises the digital three-dimensional model 1200, in addition to supplemental information. The surgical plan (and/or the digital three-dimensional model) may comprise a planned placement of the implant component 406 in the digital three-dimensional model 1200.

The computing device 102 determines the preoperative simulated performance metric by performing a preoperative kinematic analysis on the digital three-dimensional model 1200 as previously described with reference to the updated digital three-dimensional model. The preoperative kinematic analysis can comprise moving the relevant portions of the digital three-dimensional model 1200 to determine the surgical plan representing the postoperative range of motion of the joint. This movement is performed by the computing device 102 and comprises moving elements of the digital three-dimensional model 1200, such as moving bones against each other. This movement may be defined by the shape and location of bearing surfaces of joints represented by the digital three-dimensional model 1200. As previously described with reference to the kinematic analysis, the preoperative kinematic analysis may comprise a number of postoperative joint movements. Furthermore, the preoperative kinematic analysis may be associated with at least one preoperative kinematic analysis target parameter. The preoperative kinematic analysis target parameter may correspond with a respective kinematic analysis target parameter associated with the updated digital three-dimensional model.

The computing device 102 may compare the preoperative kinematic analysis with the kinematic analysis. That is, the computing device 102 may compare the preoperative kinematic analysis performed with respect to the digital three-dimensional model 1200 to the kinematic analysis performed with respect to the updated digital three-dimensional model. In some examples, the computing device 102 compares the at least one preoperative kinematic analysis target parameter with the corresponding kinematic analysis target parameter. The comparison may be used to, for example update the updated digital three-dimensional model. That is, the computing device 102 may update the updated digital three-dimensional model based on the comparison. For example, one or more of the subsequent implant components 407 may be updated based on the comparison. The update may comprise replacing the existing subsequent implant component 407 of the updated digital three-dimensional model with a different subsequent implant component 407 (e.g. of a different size, manufacturer, material and/or type), and/or may comprise updating the pose of the relevant subsequent implant component 407.

Each implant component 406 and subsequent implant component 407 size comprises unique dimensions and geometry. The progression of implant component 406 and subsequent implant component 407 dimensions for the different sized components are known. Memory 108 can therefore store features of each size of the implant component 406 and the subsequent implant components 407. The computing device 102 can compare one or more of the three-dimensional model parameters to the features of the each size the implant component 406 and subsequent implant components 407 and use the comparison to determine an optimized size of each subsequent implant component 407 and/or the implant component assembly 405 in the updated digital three-dimensional model. This comparison may be based on the risk stratification. The computing device 102 can therefore update the updated digital three-dimensional model with an optimised implant component 406 and/or an optimised supplementary implant component(s) 407. The optimised implant component 406 and/or an optimised supplementary implant component(s) 407 may be optimised by size. The optimisation may be performed with reference to the surgical parameters and/or the parameter thresholds.

Furthermore, the computing device 102 can compare one or more of the three-dimensional model parameters to the features of the each size of the implant component 406 and/or the subsequent implant components 407 and use the comparison to determine an optimized pose of each subsequent implant component 407 and/or the implant component assembly 405 in the updated digital three-dimensional model. The computing device 102 can therefore update the updated digital three-dimensional model with an optimised implant component 406 and/or an optimised supplementary implant component(s) 407. This comparison may be based on the risk stratification. The computing device 102 can update the pose of the implant component 406 and/or an supplementary implant component(s) 407 based on this optimisation in the updated digital three-dimensional model. The optimisation may performed with reference to the surgical parameters and/or the parameter thresholds.

At 310, the computing device 102 provides an indication of a clinical consequence of the pose of the localisation object 403 in relation to the surgical plan. The indication may comprise the updated surgical plan comprising the updated digital three-dimensional model. The clinical consequence may be in the form of the intraoperative simulated performance metric associated with an assessment of a placement of the implant component 406, as the placement of the implant component 406 was determined using the determined pose of the localisation object 403. In particular, the computing device 102 provides the indication of the intraoperative simulated performance metric as an assessment of a current (i.e. intraoperative) placement of the implant component 406. In providing the indication, the computing device 102 generates the indication of the intraoperative simulated performance metric. In particular, the indication module 116 generates the indication of the intraoperative simulated performance metric. The indication of the intraoperative simulated performance metric is determined as an assessment of the current placement of the implant component 406. The indication of the intraoperative simulated performance metric may also comprise an indication of the one or more alternative simulated performance metrics.

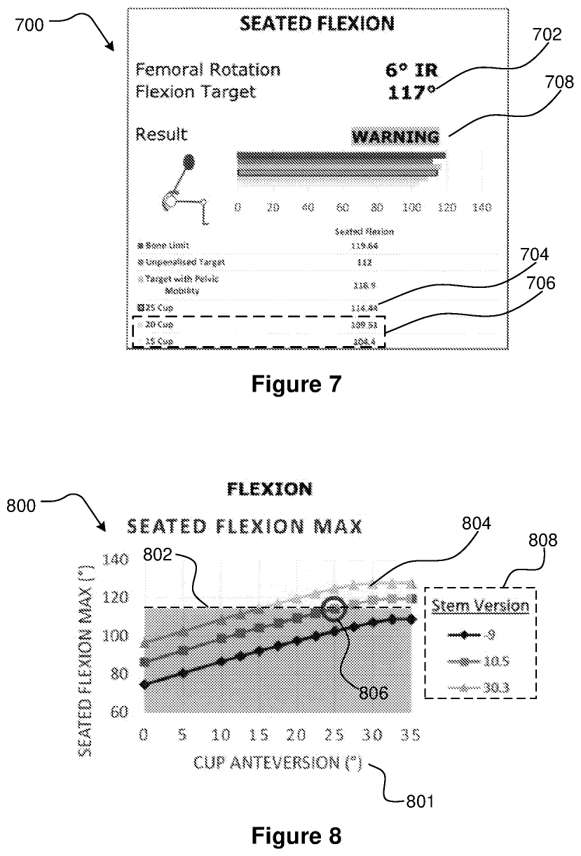

FIG. 7 illustrates an example indication 700 of the intraoperative simulated performance metric determined as an assessment of the current placement of the implant component 406. The implant component 406 under consideration in the indication 700 is the acetabular component. The indication 700 is associated with the seated flexion kinematic analysis as previously described. The indication 700 includes a intraoperative simulated performance metric 704. The intraoperative simulated performance metric 704 was determined in the kinematic analysis as previously described, and thus is an assessment of the placement of the implant component 406 based on the determined placement of the implant component 406. The indication 700 also includes a plurality of alternative simulated performance metrics 706. The indication 700 includes a kinematic analysis target parameter 702. The indication 700 includes a risk stratification 708. Thus, the indication 700 of the intraoperative simulated performance metric may be considered a risk stratification. In some examples, the risk stratification is indicative of a risk associated with multiple predicted postoperative movements by the patient. The risk stratification may be indicative of a risk of one or more of dislocation of the joint, edge loading, and postoperative joint pain. Providing the indication 700 to the surgeon may comprise displaying a graphic similar to that in FIG. 7 to the surgeon on a computer screen, or may comprise printing the graphic. The indication may also take other forms, such as a numerical score only, a bar chart as in FIG. 7, a traffic light scale (red, yellow, or green). The indication may also be audio (beep, generated voice, natural language generation) or other indicators like vibrations etc.

FIG. 8 illustrates an alternative indication 800 of the intraoperative simulated performance metric determined as an assessment of placement of the implant component 406. The indication 800 is generated in accordance with the kinematic analysis based on the updated digital three-dimensional model, and a number of alternative kinematic analyses based on alternative implant component poses, and alternative subsequent implant component 406 sizes. The indication 800 includes a kinematic analysis target parameter 802.

The indication 800 includes a plurality of simulated performance metrics 804. The simulated performance metrics 804 may comprise at least one intraoperative simulated performance metric. The simulated performance metrics 804 were determined in the kinematic analyses previously described, and thus are an assessment of the placement of the implant component 406 and selection of the subsequent implant components 407 based on the determined placement of the implant component 406. Each simulated performance metric 804 (plotted against the y-axis) is a maximum seated flexion angle. Each simulated performance metric 804 is associated with a corresponding implant component parameter 801 (the x-axis). In this case, the implant component parameter 801 is the acetabular component 406 (cup) anteversion angle. Each simulated performance metric 804 corresponds with a respective kinematic analysis performed with the particular implant component parameter 801 and subsequent implant component parameter 808. The circled simulated performance metric 806 corresponds with the kinematic analysis performed with respect to the updated digital three-dimensional model. That is, the circled simulated performance metric 806 can be considered the intraoperative simulated performance metric. Simulated performance metrics 804 above the kinematic analysis target parameter 802 represent low risk options. That is, the surgery being completed with parameters as per the simulated performance metrics 804 above the kinematic analysis target parameter 802 are less likely to result in a problematic outcome than the surgery being completed with parameters as per the simulated performance metrics 804 below the kinematic analysis target parameter 802. Thus, the indication 800 of the simulated performance metrics may be considered a risk stratification. In some examples, the risk stratification is indicative of a risk associated with multiple predicted postoperative movements by the patient. The risk stratification may be indicative of a risk of one or more of dislocation of the joint, edge loading, and postoperative joint pain. In some examples, the indication 700 and the indication 800 may be presented together as the indication of the intraoperative simulated performance metric.

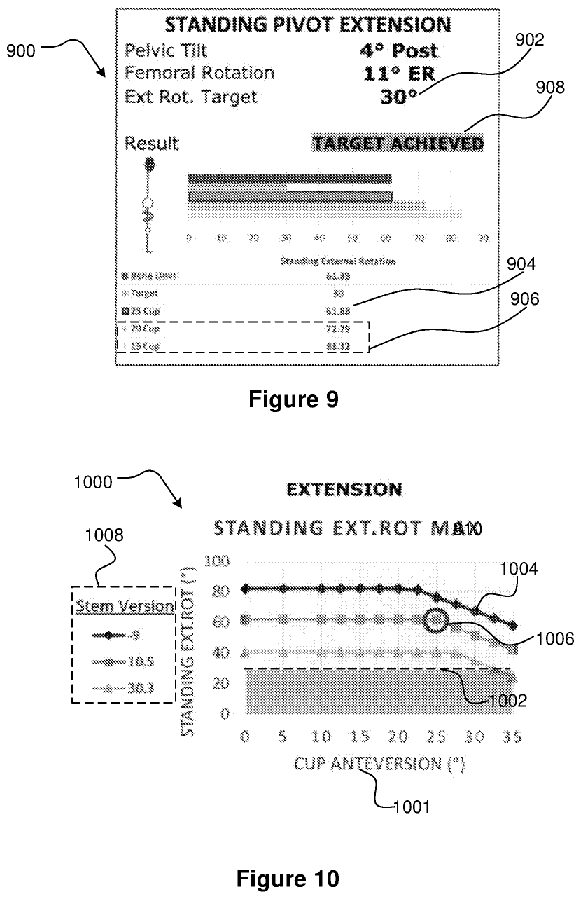

FIG. 9 illustrates another example indication 900 of the intraoperative simulated performance metric as an assessment of a placement of the implant component 406. The indication 900 is associated with the standing pivot extension kinematic analysis as previously described. The indication 900 includes an intraoperative simulated performance metric 904. The intraoperative simulated performance metric 904 was determined in the kinematic analysis previously described, and thus is an assessment of the placement of the implant component 406 based on the determined placement of the implant component 406. The indication 900 also includes a plurality of alternative simulated performance metrics 906. The indication 900 also includes an example kinematic analysis target parameter 902. The indication 900 includes a risk stratification 908. Thus, the indication 900 of the intraoperative simulated performance metric may be considered a risk stratification. In some examples, the risk stratification is indicative of a risk associated with multiple predicted postoperative movements by the patient. The risk stratification may be indicative of a risk of one or more of dislocation of the joint, edge loading, and postoperative joint pain.

FIG. 10 illustrates an alternative indication 1000 of the simulated performance metric determined as an assessment of the placement of the implant component 406. The indication 1000 is generated in accordance with the kinematic analysis based on the updated digital three-dimensional model, and a number of alternative kinematic analyses based on alternative implant component poses, and alternative subsequent implant component 407′ sizes. The indication 1000 includes a kinematic analysis target parameter 1002.

The indication 1000 includes a plurality of simulated performance metrics 1004. The simulated performance metrics 1004 were determined in the kinematic analyses previously described, and thus are an assessment of the placement of the implant component 406 and selection of the subsequent implant components 407 based on the determined placement of the implant component 406. Each simulated performance metric 1004 (plotted against the y-axis) is a maximum standing pivot extension angle as previously described. Each simulated performance metric 1004 is associated with a corresponding implant component parameter 1001 (the x-axis). In this case, the implant component parameter 1001 is the acetabular component 406 (cup) anteversion angle. Each simulated performance metric 1004 corresponds with a respective kinematic analysis performed with the particular implant component parameter 1001 and subsequent implant component parameter 1008. The circled simulated performance metric 1006 corresponds with the kinematic analysis performed with respect to the updated digital three-dimensional model. That is, the circled simulated performance metric 1006 can be considered the intraoperative simulated performance metric. Simulated performance metrics 1004 above the kinematic analysis target parameter 1002 represents low risk options. That is, the surgery being completed with parameters as per the simulated performance metrics 1004 above the kinematic analysis target parameter 1002 are less likely to result in a problematic outcome than the surgery being completed with parameters as per the simulated performance metrics 1004 below the kinematic analysis target parameter 1002. Thus, the indication 1000 of the simulated performance metrics may be considered a risk stratification. In some examples, the risk stratification is indicative of a risk associated with multiple predicted postoperative movements by the patient. The risk stratification may be indicative of a risk of one or more of dislocation of the joint, edge loading, and postoperative joint pain. In some examples, the indication 9000 and the indication 1000 may be presented together as the indication of the intraoperative simulated performance metric.

The processor 106 is configured to encode the indication of the intraoperative simulated performance metric into one or more display object(s). The display object can be in the form of a bitmap (e.g. a PNG or JPEG file) that illustrates the indication of the intraoperative simulated performance metric. Alternatively, the display object can be in the form of intraoperative simulated performance metric indication display program code executable to cause display of the indication.

The computing device 102 provides the indication of the intraoperative simulated performance metric as the assessment of the current placement of the implant component 406. In particular, the visualisation module 118 is configured to provide the indication of the intraoperative simulated performance metric. The computing device 102 displays the indication using the user interface 120. In some examples, the computing device 102 is configured to execute the performance metric indication display program code, thereby rendering the encoded indication of the intraoperative simulated performance metric on the user interface 120.

### Method 300 Performed by System 200

In some examples, the method 300, or part thereof, can be performed by a remote computing device. For example, as described below, 302, 304, 306 and 308 may be performed by the information processing device 203 that is remote from the computing device 202 and/or the imaging device 204. This can be advantageous where the computational specification(s) of the computing device 202 is insufficient to perform one or more of the steps of the method 300.

In some examples, the information processing device 203 generates the surgical plan. That is, the information processing device 203 generates the digital three-dimensional model 1200. In some examples, another computing device generates the surgical plan. That is, another computing device generates the digital three-dimensional model 1200. The digital three-dimensional model 1200 is a digital model. The digital three-dimensional model 1200 may be a hip, knee, shoulder, elbow or another joint. The digital three-dimensional model 1200 can comprise an anatomical three-dimensional model 1202 and an implant component assembly three-dimensional model 1204, and can be generated as previously described with reference to method 300 being performed by system 100. That is, the digital three-dimensional model 1200 is generated using information provided by the preoperative imaging device, and is a three-dimensional model of the joint to be replaced in the joint replacement surgery. Furthermore, as previously described, the implant component assembly three-dimensional model 1204 is a three-dimensional representation of the implant component assembly 405.

In some examples, the preoperative imaging device may be configured to provide the information to the processor 206. The processor 206 processes the information provided by the preoperative imaging device to generate the anatomical three-dimensional model 1202. The anatomical three-dimensional model 1202 can be stored in memory 208.

In some examples, a model generating computing device (not shown) processes the information provided by the preoperative imaging device to generate the anatomical three-dimensional model 1202. In said examples, the anatomical three-dimensional model 1202 can be provided to the information processing device 203. The anatomical three-dimensional model 1202 can be stored in memory 208.

The digital three-dimensional model 1200 represents the intended joint configuration after the surgery by comprising the anatomical three-dimensional model 1202 and the implant component assembly three-dimensional model 1204.

In some examples the information processing device 203 processes the digital three-dimensional model 1200 as described with reference to method 300 being performed by system 100. In some examples, the model generating computing device or another computing device processes the digital three-dimensional model 1200 and transmits the processed digital three-dimensional model 1200 to the information processing device 203. Processing the digital three-dimensional model 1200 may comprise scaling the digital three-dimensional model 1200 and/or determining one or more digital three-dimensional model parameters, landmarks and/or measurements as previously described. In some examples, the digital three-dimensional model parameters may comprise anatomical three-dimensional model parameters as previously described. In some examples, digital three-dimensional model parameters may comprise implant component assembly three-dimensional model parameters as previously described.

At 302, the information processing device 203 stores surgical plan in memory 208. That is, the information processing device 203 stores the digital three-dimensional model 1200 in memory 208. The information processing device 203 stores the digital three-dimensional model 1200, and the associated digital three-dimensional model parameters.

The imaging device 204 captures the digital X-ray image 1100 of the joint and the localisation object 403. In particular, the imaging device 204 captures the digital X-ray image 1100 of the joint and the localisation object 403 during the total joint replacement surgery. In some examples, the digital X-ray image 1100 is an intraoperative X-ray image of a patient's hip as previously described.

At 304, the information processing device 203 receives digital X-ray image 1100 of the joint and the localisation object 403. The processor 206 stores the digital X-ray image 1100 of the joint and the localisation object 403 in memory 208. In some examples, the imaging device 204 transmits the digital X-ray image 1100 to the information processing device 203 over the communications network 250.

The information processing device 203 processes the digital X-ray image 1100. Processing the digital X-ray image 1100 may comprise determining one or more digital image parameters as described with reference to method 300 being performed by system 100. The one or more digital image parameters may comprise locations of one or more digital image landmarks as previously described. Each digital image landmark may have a determined digital image landmark location as previously described. The digital image parameters may comprise one or more digital image measurements as previously described. For example, the digital image measurements are indicative of a distance between two or more digital image landmarks.

One or more of the digital image parameters may correspond with one or more of the digital three-dimensional model parameters. Therefore, one or more of the digital image landmarks may correspond with a respective three-dimensional model landmark. Furthermore, one or more of the digital image measurements may correspond with a respective three-dimensional model measurement.

In some examples, processing the digital X-ray image 1100 comprises scaling the digital X-ray image 1100. The digital X-ray image 1100 may be scaled as described with reference to method 300 being performed by system 100.

In some examples, processing the digital X-ray image 1100 comprises detecting one or more edges in the digital X-ray image 1100. For example, the information processing device 203 detects the edges of the localisation object 403. The information processing device 203 may detect the edges using a suitable edge detection method, such as using a Sobel operator.

In some examples, processing the digital X-ray image 1100 comprises detecting one or more objects in the digital X-ray image 1100. For example, an anatomical feature, e.g. a bone, may be detected in the digital X-ray image 1100. Furthermore, one or more of the localisation object 403, implant component 406 and/or subsequent implant components 407 may be detected in the digital X-ray image 1100. In particular, the implant component 406 may be detected in the digital X-ray image 1100. Alternatively, the localisation object 403 can be detected in the digital X-ray image 1100.

The information processing device 203 detects the objects (e.g. the localisation object 403) in the digital X-ray image 1100. The information processing device 203 may detect the objects in the digital X-ray image 1100 as described with reference to method 300 being performed by system 100.

In some examples, the information processing device 203 also determines the pose of the objects in the digital X-ray image 1100. For example, after detecting the implant component 406, the information processing device 203 is configured to determine the pose of the implant component 406. The information processing device 203 may be configured to determine the pose of the implant component 406 as described with reference to method 300 being performed by system 100. The pose of the implant component 406 may comprise an indication of the location and orientation of the implant component 406.

In some examples, the information processing device 203 uses the detected edges, objects and/or poses of said objects to determine the one or more digital image parameters.

At 306, the information processing device 203 determines the pose of the localisation object 403. The information processing device 203 determines the pose of the localisation object relative to the bone or the joint based on the digital X-ray image 1100. In particular, the pose determination module 212 determines the pose of the localisation object 403. The pose of the localisation object 403 is determined relative to the bone or the joint based on the digital X-ray image 1100.

The information processing device 203 may have stored an object model of the localisation object 403. The information processing device 203 may match the object model against objects identified in the digital X-ray image 1100 as previously described. For example, the information processing device 203 fits a localisation object model to the localisation object 403. The information processing device 203 then determines the position and pose of the localisation object 403.

The imaging device 204 may be fixed in relation to the operating table, with a known viewing angle and distance. Thus, the information processing device 203 can calculate the position and pose of the localisation object 403 in relation to this global reference frame. Processor 206 may implement feature detection using Haar-like features is disclosed in Viola and Jones, “Rapid object detection using a boosted cascade of simple features”, Computer cool Vision and Pattern Recognition, 2001, which is incorporated herein by reference.

In another example, the information processing device 203 is configured to detect one or more bones in the digital X-ray image 1100 as previously described with reference to the computing device 102.

In another example, the information processing device 203 is configured to detect the implant component 406 and/or one or more of the subsequent implant components 407 as previously described with reference to the computing device 102.

Once the bone, implant component 406 or subsequent implant component 407 is identified in the digital X-ray image 1100, the information processing device 203 can determine the relative position and pose of the bone, implant component 406 or subsequent implant component 407 in relation to the localisation object 403. The relative position may be expressed as three offset angles and three translation values. Furthermore, as the location and pose of the localisation object 403 is known in the global reference frame, the location and pose of each of the implant component 406, subsequent implant component 407 and/or bone can be determined.

In some examples, the localisation object 403 is directly connected to the implant component 406. The localisation object 403 can be connected to the implant component 406 at a fixed position. The connection can be a rigid connection. This is the case illustrated in FIG. 4. Therefore, processor 206 has available a fixed spatial relationship between the localisation object 403 and the implant component 406, such as three offset angles and three offset coordinates. Thus, by determining the position and pose of the localisation object 403, the information processing device 203 can determine the corresponding position and pose of the implant component 406. The pose of the localisation object 403 can therefore be considered to be associated with an implant component pose. This can advantageously be done without the information processing device 203 having to detect the implant component 406 in the digital X-ray image 1100, reducing computational requirements of the information processing device 203.

In some examples, the localisation object 403 is directly connected to the subsequent implant component 407. Therefore, processor 206 has available a fixed spatial relationship between the localisation object 403 and the subsequent implant component 407, such as three offset angles and three offset coordinates. Thus, by determining the position and pose of the localisation object 403, the computing device 102 can determine the corresponding position and pose of the subsequent implant component 407 as previously described. The pose of the localisation object 403 can therefore be considered to be associated with a subsequent implant component pose.

In some examples, the localisation object 403 is directly connected to the relevant bone. Therefore, processor 206 has available a fixed spatial relationship between the localisation object 403 and the bone, such as three offset angles and three offset coordinates. Thus, by determining the position and pose of the localisation object 403, the computing device 102 can determine the corresponding position and pose of the bone (e.g. the femur 404 or the pelvis 402) as previously described. The pose of the localisation object 403 can therefore be considered to be associated with a bone pose.

In some examples, the implant component 406 may comprise the localisation object 403. Alternatively, the implant component 460 may be the localisation object 403. For example, the implant component 406 may comprise an X-ray opaque two-dimensional code on a surface of the implant component 406. This may be an Aruco code and processor 206 may execute an Aruco library available at https://docs.opencv.org/trunk/d9/d6a/group_aruco.html. This enables processor 206 to identify the location and pose of the localisation object 403 without object detection as previously described.

At 308, the information processing device 203 assesses the pose of the localisation object 403 against the surgical plan. In some examples, the information processing device 203 may assess the pose of the implant component 406 against the surgical plan. As the pose of the implant component 406 was determined using the determined pose of the localisation object 403, this can be considered an assessment of the pose of the localisation object 403 against the surgical plan. In some examples, the information processing device 203 may assess the pose of the subsequent implant component 407 against the surgical plan. As the pose of the subsequent implant component 407 was determined using the determined pose of the localisation object 403, this can be considered an assessment of the pose of the localisation object 403 against the surgical plan. In some examples, the information processing device 203 may assess the pose of the bone (e.g. the pelvis 404 or femur 402) against the surgical plan. As the pose of the bone was determined using the determined pose of the localisation object 403, this can be considered an assessment of the pose of the localisation object 403 against the surgical plan. Although described below with reference to the implant component 406, it will be appreciated that the description can also be applied to the subsequent implant component 407 and/or the bone.

In some examples, the information processing device 203 determines one or more differences between the pose of the implant component 406 as represented in the digital X-ray image 1100 (and determined using the localisation object 403), and the pose of the implant component 406 of the digital three-dimensional model 1200.

In some examples, the information processing device 203 compares one or more of the digital image parameters to one or more parameter thresholds. The parameter thresholds can be indicative of the desired surgical parameters, or acceptable surgical parameters. For example, in the case of the total hip replacement, a parameter threshold can be an implant component inclination angle threshold of 40°. That is, the desired inclination angle of the implant component is 40°. The inclination angle of the implanted implant component can be determined from the digital X-ray image 1100 as previously described, and this can be compared to the implant component inclination angle threshold. In some examples, implant component inclination angle threshold is a range, for example, between 30° and 50°. The surgeon may specify the parameter thresholds, which may be selected to maximise the postoperative performance of the joint. Alternatively, the information processing device 203 can automatically determine the parameter thresholds. If the implant component 406 is determined to deviate from its corresponding parameter thresholds, it can be classified as high risk.

In some examples, the parameter thresholds are equal to the desired surgical parameters. In other examples, the parameter thresholds are threshold ranges centred upon, or including the desired surgical parameter.

The information processing device 203 may determine an updated digital three-dimensional model. The information processing device 203 updates the pose of the implant component 406 in the digital three-dimensional model 1200 to reflect the actual implanted pose of the implant component 406 determined from the digital X-ray image 1100 using the localisation object 403. Thus, the digital three-dimensional model 1200 is intraoperatively updated to reflect the state of the surgery at the time the digital X-ray image 1100 was captured. Updating the pose of the implant component 406 may comprise, for example, translating and/or rotating the implant component 406 of the digital three-dimensional model 1200. The information processing device 203 updates the digital three-dimensional model 1200 based on the determined placement of the implant component 406 in the digital X-ray image 1100 in relation to the digital three-dimensional model 1200, thereby determining the updated digital three-dimensional model.

The information processing device 203 determines an intraoperative simulated performance metric by simulating movement of the updated digital three-dimensional model based on the placement of the implant component 406 (determined using the localisation object 403) in the digital X-ray image 1100. In particular, the performance metric simulation module 114 determines the intraoperative simulated performance metric by simulating movement of the updated digital three-dimensional model.

The information processing device 203 determines the intraoperative simulated performance metric by performing a kinematic analysis on the updated digital three-dimensional model. The kinematic analysis can comprise moving the relevant portions of the updated digital three-dimensional model to determine a postoperative range of motion of the joint. This movement is performed by the information processing device 203 and comprises moving elements of the digital three-dimensional model 1200, such as moving bones against each other. This movement may be defined by the shape and location of bearing surfaces of joints represented by the updated digital three-dimensional model.

The kinematic analysis performed by the information processing device 203 may be as described with reference to system 100 and at least FIGS. 5 and 6. That is, the kinematic analysis may comprise a number of postoperative joint movements. Each postoperative joint movement can simulate a typical movement of the patient after the surgery. The kinematic analysis may comprise the seated flexion movement and/or the standing pivot extension movement as previously described.

As previously described, the kinematic analysis is associated with at least one kinematic analysis target parameter. Each kinematic analysis target parameter can be indicative of a desired or target performance of the joint. For example, the kinematic analysis target parameter can be an angle representing a target rotation desired of the joint before an impingement occurs. The information processing device 203 is configured to provide a risk stratification based on a comparison between the kinematic performance of the updated digital three-dimensional model and the at least one kinematic analysis target parameter.

In some examples, a flexion target parameter can be associated with the seated flexion movement of the kinematic analysis as described with reference to system 100. Furthermore, an extension rotation target parameter can be associated with the standing pivot extension of the kinematic analysis as described with reference to system 100.

The information processing device 203 may also compare a current (i.e. intraoperative) implant component pose with a number of alternative poses (e.g. of the acetabular component) by determining an alternative simulated performance metric associated with an alternative implant component pose. In other words, the information processing device 203 may compare an intraoperative implant component pose with the number of alternative poses. The information processing device 203 can adjust the pose of the implant component 406 in the updated digital three-dimensional model, and re-run the kinematic analysis. The information processing device 203 is configured to provide an alternative risk stratification based on a comparison between the kinematic performance of the updated digital three-dimensional model with the alternative implant component pose and the at least one kinematic analysis target parameter. For example, the information processing device 203 can change the acetabular inclination angle of the implant component 406, and re-run the kinematic analysis. In some examples, this can be used to assist the surgeon in determining whether or not the implant component 406 that has been implanted should be removed, and/or re-implanted in a different position as previously described.

In some examples, the information processing device 203 also determines the alternative simulated performance metric associated with an alternative subsequent implant component 407′. As previously described, the updated digital three-dimensional model includes one or more subsequent implant components 407 that are to be implanted after the implant component 406. The positioning of the implant component 406 that has been implanted may however mean the originally planned subsequent implant components 407 are unsuitable. Thus, the information processing device 203 determines the alternative simulated performance metric associated with the alternative subsequent implant component 407′. The alternative simulated performance metric can be compared to the intraoperative simulated performance metric to assess surgical options. In some examples, this can be used to assist the surgeon in intraoperatively determining appropriate sizing for the subsequent implant components 407.

The information processing device 203 determines the alternative subsequent implant component 407′. The computing device 102 can substitute the alternative subsequent implant component 407′ for the subsequent implant component 407 in the updated digital three-dimensional model, and re-run the kinematic analysis. The information processing device 203 is configured to provide an alternative risk stratification based on a comparison between the kinematic performance of the updated digital three-dimensional model with the subsequent implant component 407 and the alternative subsequent implant component 407′ using the kinematic analysis target parameter.

In some examples, the information processing device 203 determines a preoperative simulated performance metric. The information processing device 203 determines the preoperative simulated performance metric by simulating movement of the digital three-dimensional model 1200 according to a surgical plan. In some examples, the surgical plan is the digital three-dimensional model 1200. In some examples, the surgical plan comprises the digital three-dimensional model 1200, in addition to supplemental information. The surgical plan (and/or the digital three-dimensional model) may comprise a planned placement of the implant component in the digital three-dimensional model 1200.

The information processing device 203 determines the preoperative simulated performance metric by performing a preoperative kinematic analysis on the digital three-dimensional model as previously described with reference to the updated digital three-dimensional model. The preoperative kinematic analysis can comprise moving the relevant portions of the digital three-dimensional model 1200 to determine the surgical plan representing the postoperative range of motion of the joint. This movement is performed by the information processing device 203 and comprises moving elements of the digital three-dimensional model, such as moving bones against each other as described with reference to the method 300 being performed by the system 100. T

As previously described with reference to the method 300 being performed by the system 100, the preoperative kinematic analysis may be associated with at least one preoperative kinematic analysis target parameter. The preoperative kinematic analysis target parameter may correspond with a respective kinematic analysis target parameter associated with the updated digital three-dimensional model.

The information processing device 203 may compare the preoperative kinematic analysis with the kinematic analysis. That is, the information processing device 203 may compare the preoperative kinematic analysis performed with respect to the digital three-dimensional model 1200 to the kinematic analysis performed with respect to the updated digital three-dimensional model. In some examples, the information processing device 203 compares the at least one preoperative kinematic analysis target parameter with the corresponding kinematic analysis target parameter. The comparison may be used to, for example update the updated digital three-dimensional model. That is, the information processing device 203 may update the updated digital three-dimensional model based on the comparison. For example, one or more of the subsequent implant components 407 may be updated based on the comparison. The update may comprise replacing the existing subsequent implant component 407 of the updated digital three-dimensional model with a different subsequent implant component 407 (e.g. of a different size, manufacturer, material and/or type), and/or may comprise updating the pose of the relevant subsequent implant component 407.

Each implant component 406 and subsequent implant component 407 size comprises unique dimensions and geometry. The progression of implant component 406 and subsequent implant component 407 dimensions for the different sized components are known. Memory 208 can therefore store features of each size of the implant component 406 subsequent implant component 407. The information processing device 203 can compare one or more of the three-dimensional model parameters to the features of each size of the implant component 406 and/or the subsequent implant components 407 and use the comparison to determine an optimized size of each subsequent implant component 407 and/or the implant component assembly 405 in the updated digital three-dimensional model. This comparison may be based on the risk stratification. The information processing device 203 can therefore update the updated digital three-dimensional model with an optimised implant component 406 and/or an optimised supplementary implant component(s) 407. The optimised implant component 406 and/or an optimised supplementary implant component(s) 407 may be optimised by size. The optimisation may be performed with reference to the surgical parameters and/or the parameter thresholds.

Furthermore, the information processing device 203 can compare one or more of the three-dimensional model parameters to the features of each size of the implant component 406 and/or the subsequent implant components 407 and use the comparison to determine an optimized pose of each subsequent implant component 407 and/or the implant component assembly 405 in the updated digital three-dimensional model. This comparison may be based on the risk stratification. The information processing device 203 can therefore update the updated digital three-dimensional model with an optimised implant component 406 and/or an optimised supplementary implant component(s) 407. The information processing device 203 can update the pose of the implant component 406 and/or an supplementary implant component(s) 407 based on this optimisation in the updated digital three-dimensional model. The optimisation may performed with reference to the surgical parameters and/or the parameter thresholds.

At 310, the information processing device 203 provides an indication of a clinical consequence of the pose of the localisation object 403 in relation to the surgical plan. The indication may comprise the updated surgical plan comprising the updated digital three-dimensional model. The clinical consequence may be in the form of the intraoperative simulated performance metric associated with an assessment of a placement of the implant component 406, as the placement of the implant component 406 was determined using the determined pose of the localisation object 403. In particular, the information processing device 203 provides the indication of the intraoperative simulated performance metric as an assessment of a current (i.e. intraoperative) placement of the implant component. In providing the indication, the information processing device 203 generates an indication of the intraoperative simulated performance metric. In particular, the indication module 216 generates the indication of the intraoperative simulated performance metric. The indication of the intraoperative simulated performance metric is determined as an assessment of a placement of the implant component 406. The indication of the intraoperative simulated performance metric may also comprise an indication of the one or more alternative simulated performance metrics.

The information processing device 203 may generate an indication 700 of the intraoperative simulated performance metric as described with reference to system 100 and FIGS. 7, 8, 9 and/or 10.

The processor 206 is configured to encode the indication of the intraoperative simulated performance metric into one or more display object(s). The display object can be in the form of a bitmap (e.g. a PNG or JPEG file) that illustrates the indication of the intraoperative simulated performance metric. Alternatively, the display object can be in the form of intraoperative simulated performance metric indication display program code executable to cause display of the indication. The information processing device 203 is configured to transmit the one or more display objects to the computing device 202 using the communications network 250.

The computing device 202 provides the indication of the intraoperative simulated performance metric as the assessment of a placement of the implant component 406. The computing device 202 is configured to execute the performance metric indication display program code, thereby rendering the encoded indication of the intraoperative simulated performance metric on the user interface 120.

### Advantages

As previously described, surgeons can modify a large number of parameters in surgeries, and in particular, in joint replacement surgeries. The disclosed examples enable the surgeon to intraoperatively assess the progress of the surgery, and continue, or adjust the course of the surgery in accordance with feedback provided by the disclosed examples.

By generating and storing the digital three-dimensional model 1200 of the joint, the surgeon has available a detailed surgical plan that can be used as a target outcome for the surgery. Intraoperatively capturing the digital X-ray image 1100 enables intraoperative analysis of surgical progress. The pose of the localisation object can be accurately determined from a two-dimensional projection of the localisation object. That is, the profile of the localisation object in a two-dimensional image (e.g. the described digital image) can be used to accurately determine its pose. As there is a direct relationship between the pose of the localisation object and the pose of the implant component, this can allow accurate determination of the pose of the implant component from the digital image. This is, for example allowed by the direct connection between the implant component and the localisation object. That is, movement of the implant component imparts corresponding movement to the localisation object. The same advantages apply when the localisation object is connected to one of the bones of the joint, or one of the subsequent implant components.

Incorrectly implanting the implant component 406 can result in a number of undesirable postoperative outcomes. For example, in total hip replacements, incorrect acetabular cup positioning can increase the risk of postoperative joint dislocations, edge loading and joint pain. Postoperative joint dislocations cause great discomfort to the patient, and can require subsequent surgical intervention. Edge loading can cause premature wear of the joint. Joint pain again causes discomfort to the patient.

In the disclosed examples, the digital three-dimensional model 1200 is updated based on the determined pose of the localisation object (and thus, the implant component, subsequent implant component and/or bone) to more accurately reflect the current operative state. This enables simulation and optimisation of the performance of the joint.

In some examples, subsequent implant components of the updated digital three-dimensional model can also be optimised and updated. The optimisation can be performed with reference to the surgical parameters and/or the parameter thresholds and can thus improve the outcome of the surgery by increasing the likelihood that the final joint will fall within the surgical parameters and/or the parameter thresholds.

The disclosed kinematic analysis is used to determine the intraoperative simulated performance metric of the joint based on the updated digital three-dimensional model. The intraoperative simulated performance metric is provided to the surgeon, and provides the surgeon with an insight into the future performance or the joint during the operation. Where the intraoperative simulated performance metric indicates there is a high risk of an undesirable postoperative outcome, the surgeon may adjust one or more of the surgical parameters accordingly to attempt to improve it. For example, the surgeon may attempt to reposition the implant component. Alternatively, the surgeon may select alternative subsequent implant components to compensate for the state of the implant component that has already been implanted.

Some examples pre-operatively support the surgeon's decision making process by performing the kinematic analysis across a range of implant component poses, and subsequent implant component sizes. The results of this analysis may be presented to the surgeon in the form of a risk stratification. Furthermore, some examples can determine optimised parameters, and make corresponding suggestions to the surgeon. For example, some examples can suggest optimised subsequent implant component sizes that minimise the risk of postoperative complications.

It will be appreciated by persons skilled in the art that numerous variations and/or modifications may be made to the above-described examples, without departing from the broad general scope of the present disclosure. The present examples are, therefore, to be considered in all respects as illustrative and not restrictive.

It will be appreciated by persons skilled in the art that numerous variations and/or modifications may be made to the specific examples without departing from the scope as defined in the claims.

It should be understood that the techniques of the present disclosure might be implemented using a variety of technologies. For example, the methods described herein may be implemented by a series of computer executable instructions residing on a suitable computer readable medium. Suitable computer readable media may include volatile (e.g. RAM) and/or non-volatile (e.g. ROM, disk) memory, carrier waves and transmission media. Exemplary carrier waves may take the form of electrical, electromagnetic or optical signals conveying digital data streams along a local network or publically accessible network such as the internet.

It should also be understood that, unless specifically stated otherwise as apparent from the following discussion, it is appreciated that throughout the description, discussions utilizing terms such as “estimating” or “processing” or “computing” or “calculating”, “optimizing” or “determining” or “displaying” or “maximising” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

The present examples are, therefore, to be considered in all respects as illustrative and not restrictive.

1. An intraoperative localisation system for total joint replacement of a joint of a patient by a surgeon, the joint being associated with a bone, the localisation system comprising:
an X-ray imaging device for application of X-ray radiation to the joint and for detecting X-ray radiation to create a digital X-ray image of the joint and a localisation object during a total joint replacement surgery;
a computer system configured to:
store a surgical plan comprising a digital three-dimensional model;
receive the digital X-ray image of the joint and the localisation object during the total joint replacement surgery;
determine a pose of the localisation object relative to the bone or the joint, based on the digital X-ray image;
assess the pose of the localisation object against the surgical plan; and
provide an indication of a clinical consequence of the pose in relation to the surgical plan to the surgeon.

2. The system of claim 1, wherein the pose is associated with a bone pose.

3. The system of claim 1, wherein the pose is associated with implant component pose.

4. The system of any one of claims 1 to 3, further comprising the localisation object.

5. The system of any one of claims 1 to 4, wherein the indication comprises an updated surgical plan.

6. The system of claim 5, wherein the updated surgical plan comprises an updated digital three-dimensional model.

7. The system of any one of claims 1 to 6, wherein the computer system is configured to determine a preoperative simulated performance metric by simulating movement of the digital three-dimensional model.

8. The system of claim 6, wherein the computer system is configured to determine an intraoperative simulated performance metric by simulating movement of the updated digital three-dimensional model.

9. The system of claim 8, wherein the indication comprises the intraoperative simulated performance metric.

10. The system of any one of claims 1 to 9, wherein the indication comprises a comparison between the intraoperative simulated performance metric and the preoperative simulated performance metric.

11. The system of any one of claims 8 to 10, wherein the intraoperative simulated performance metric is an indication of a risk stratification.

12. The system of claim 11, wherein the risk stratification is indicative of a risk associated with multiple predicted postoperative movements by the patient.

13. The system of claim 11 or 12, wherein the risk stratification is indicative of a risk of one or more of:
dislocation of the joint;
edge loading; and
postoperative joint pain.

14. The system of any one of claims 1 to 13, wherein determining the pose of the localisation object comprises detecting objects in the digital image and fitting an object model to the objects.

15. The system of any one of claims 1 to 14, wherein the localisation object comprises an X-ray opaque two-dimensional code, and wherein determining the pose of the localisation object comprises determining a pose associated with the X-ray opaque two-dimensional code.

16. The system of any one of claims 1 to 15, wherein the computer system comprises:
at least one processor; and
at least one memory storing program code accessible by the at least one processor, and configured to cause the at least one processor to:
store the surgical plan;
receive the digital X-ray image of the joint and the localisation object during the total joint replacement surgery;
determine the pose of the localisation object relative to the bone or the joint based on the digital X-ray image;
assess the pose of the localisation object against the surgical plan; and
providing the indication to the surgeon.

17. The system of any one of claims 1 to 15, wherein the computer system comprises:
a first computing device comprising:
at least one first processor; and
at least one first memory storing program code accessible by the at least one first processor, and configured to cause the at least one first processor to:
store the store the surgical plan;
receive the digital X-ray image of the joint and the localisation object during the total joint replacement surgery;
determine the pose of the localisation object relative to the bone or the joint based on the digital X-ray image; and
assess the pose of the localisation object against the surgical plan; and
a second computing device comprising:
at least one second processor; and
at least one second memory storing program code accessible by the at least one second processor, and configured to cause the at least one second processor to:
provide the indication to the surgeon.

18. The system of claim 16, wherein the computing device is configured to receive the digital X-ray image from an X-ray imaging device.

19. The system of claim 17, wherein the second computing device is configured to receive the digital X-ray image from an X-ray imaging device.

20. The system of any one of claims 1 to 19, wherein the system comprises a display, and wherein the indication is provided as a visual output using the display.

21. The system of any one of claims 1 to 20, wherein determining the pose of the localisation object comprises identifying one or more edges of the localisation object in the digital X-ray image.

22. The system of any one of claims 1 to 21, wherein the joint is a hip joint.

23. A computer-implemented method for assisting a surgeon in total joint replacement of a joint of a patient, the method comprising:
storing a surgical plan comprising a digital three-dimensional model;
receiving a digital X-ray image of the joint and a localisation object during a total joint replacement surgery;
determining a pose of the localisation object relative to a bone or the joint, based on the digital X-ray image;
assessing the pose of the localisation object against the surgical plan; and
providing an indication of a clinical consequence of the pose in relation to the surgical plan to the surgeon.

24. The method of claim 23, wherein the pose is associated with a bone pose.

25. The method of claim 23, wherein the pose is associated with an implant component pose.

26. The method of any one of claims 23 to 25, wherein the indication comprises an updated surgical plan.

27. The method of claim 26, wherein the updated surgical plan comprises an updated digital three-dimensional model.

28. The method of any one of claims 23 to 27, further comprising determining a preoperative simulated performance metric by simulating movement of the digital three-dimensional model.

29. The method of claim 27, further comprising determining an intraoperative simulated performance metric by simulating movement of the updated digital three-dimensional model.

30. The method of claim 29, wherein the indication comprises the intraoperative simulated performance metric.

31. The method of any one of claims 23 to 30, wherein the indication comprises a comparison between the intraoperative simulated performance metric and the preoperative simulated performance metric.

32. The method of any one of claims 29 to 31, wherein the intraoperative simulated performance metric is an indication of a risk stratification.

33. The method of claim 32, wherein the risk stratification is indicative of a risk associated with multiple predicted postoperative movements by the patient.

34. The method of claim 32 or claim 33, wherein the risk stratification is indicative of a risk of one or more of:
dislocation of the joint;
edge loading; and
postoperative joint pain.

35. The method of any one of claims 23 to 34, wherein determining the pose of the localisation object comprises detecting objects in the digital X-ray image and fitting an object model to the objects.

36. The method of any one of claims 23 to 35, wherein determining the pose of the localisation object comprises determining a pose associated with an X-ray opaque two-dimensional code.

37. The method of any one of claims 23 to 36, further comprising providing the indication as a visual output using a display.

38. The method of any one of claims 23 to 37, wherein determining the pose of the localisation object comprises identifying one or more edges of the localisation object in the digital X-ray image.

39. The method of any one of claims 23 to 38, wherein the joint is a hip.

40. A computer-readable storage medium storing instructions that, when executed by a computing device, cause the computing device to perform the method of any one of claims 23 to 39.
